(19) United States
(12) Patent Application Publication
(10) Pub. No.: US 2024/0301090 A1
(43) Pub. Date: Sep. 12, 2024

(54) COMPOSITIONS AND METHODS FOR TREATING A MEDICAL CONDITION

(71) Applicant: Technion Research & Development Foundation Limited, Haifa (IL)

(72) Inventors: Philippa MELAMED, Haifa (IL); Lilach Pnueli POLINSKY, Haifa (IL)

(73) Assignee: Technion Research & Development Foundation Limited, Haifa (IL)

(21) Appl. No.: 18/578,329

(22) PCT Filed: Jul. 13, 2022
(86) PCT No.: PCT/IL2022/050752

§ 371 Date: Jan. 11, 2024

Related U.S. Application Data
(60) Provisional application No. 63/221,056, filed on Jul. 13, 2021.

(51) Int. Cl. C07K 19/00; A61K 38/00; C07K 7/23

(52) U.S. Cl. CPC C07K 19/00; C07K 7/23; A61K 38/00; C07K 2319/10; C07K 2319/50; C07K 2319/80

(57) ABSTRACT

Compositions and methods for treating a medical condition are provided. Accordingly, there is provided a composition comprising a polypeptide comprising a GnRH receptor binding protein and a polynucleotide coding a therapeutic agent, wherein said polypeptide is in association with said polynucleotide via a linker comprising a cleavage motif that can be cleaved by a proteolytic enzyme secreted by a cell expressing said GnRH receptor. Also provided is a nucleic acid construct comprising a nucleic acid sequence encoding a fusion protein comprising a DNA binding domain (DBD) of a transcription factor that binds a regulatory region of a gonadotropin gene attached to a chromatin modifying domain that represses transcription upon association with the DBD. Also provided are methods of use thereof.

### RELATED APPLICATION

This application claims the benefit of priority of U.S. Patent Application No. 63/221,056 filed on Jul. 13, 2021, the contents of which are incorporated herein by reference in their entirety.

### SEQUENCE LISTING STATEMENT

The ASCII file, entitled 92631 SequenceListing.txt, created on Jul. 12, 2022, comprising 42,002 bytes, submitted concurrently with the filing of this application is incorporated herein by reference.

### FIELD AND BACKGROUND OF THE INVENTION

The present invention, in some embodiments thereof, relates to compositions and methods for treating a medical condition.

Treatment of multiple diseases include administration of drugs that regulate gene expression, induce cytotoxic or apoptotic effects, enable production of a protein or a peptide and the like. While drug delivery takes a variety of forms, depending on the disease, the agent to be delivered and the administration route; targeted delivery and techniques enabling efficient transfer of a drug into a target cell are of main interest. Targeted drug delivery (e.g., to a particular cell type; to a diseased cell but not to a healthy cell; etc.) minimize toxic side effects, lower the required dosage amounts, localize drug release and delivery, and decrease costs for the patient.

Gonadotrophin releasing hormone (GnRH) receptors are G-protein-coupled receptors expressed primarily in the pituitary gonadotropes. These receptors mediate the effects of the hypothalamic hormone GnRH on the pituitary, to activate the reproductive axis. Due to the role of GnRH in driving reproductive function, steroid-dependent cancers (e.g. breast, endometrial and prostate) are routinely treated with drugs to target this receptor to repress the reproductive axis thus also production of these steroids. However evidence exits showing expression of GnRH and its receptor in extra-pituitary tissue as well as in numerous human cancers, including virtually all breast, prostate, ovarian and endometrial cancers, as well as those not related to reproductive tissues, such as bladder, lung, melanoma, leukemia, glioblastoma and pancreas [Maggi, R. et al. Hum. Reprod. Update 22, 358-381 (2016); Grindker, C. & Emons, G. Front. Endocrinol. (Lausanne). 8, (2017)]. Hence, GnRH-targeted approaches were suggested for delivery of toxins to GnRH receptor-expressing tumor cells [see e.g. Grindker, C., et al. Oncol. Rep. 2011, 26, 629-635; Li, X., et al. Mini-Reviews Med. Chem. 17, 258-267 (2016); B. Engel, J., et al. Curr. Drug Targets 17, 488-494 (2016); Miller, D. S. et al. J. Clin. Oncol. 36, 5503-5503 (2018); Davenport, A. P., et al. Nature Reviews Drug Discovery 19, 389-413 (2020); Limonta P, et al. Endocr Rev. 2012, 33(5):784-811; Curtis K K, et al. Cancer Chemother Pharmacol. 2014, 73(5):931-941; FOst, C., et al. Oncol. Rep. 2011, 25, 1481-1487; Seitz, S., et al. BMC Cancer 2014, 14; Kwok, C. W., et al. Target. Oncol. 2015, 10, 365-373; Westphalen, S., et al. Int. J. Oncol. 2000, 17, 1063-1069]. Additional background art includes:
U.S. Pat. No. 8,821,943 and U.S. Ser. No. 10/704,038; and
Japanese Patent No. JP6817288.

### SUMMARY OF THE INVENTION

According to an aspect of some embodiments of the present invention there is provided a composition comprising a polypeptide comprising a GnRH receptor binding protein and a polynucleotide coding a therapeutic agent, wherein the polypeptide is in association with the polynucleotide via a linker comprising a cleavage motif that can be cleaved by a proteolytic enzyme secreted by a cell expressing the GnRH receptor.

According to some embodiments of the invention, the polynucleotide is attached to—or encapsulated by a cell penetrating moiety.

According to an aspect of some embodiments of the present invention there is provided a method of treating a medical condition in a subject in need thereof, the method comprising administering to the subject a therapeutically effective amount of the composition, wherein cells associated with the medical condition express the GnRH receptor and secrete the proteolytic enzyme, thereby treating the medical condition in the subject.

According to an aspect of some embodiments of the present invention there is provided the composition, for use in treating a medical condition in a subject in need thereof, wherein cells associated with the medical condition express the GnRH receptor and secrete the proteolytic enzyme.

According to some embodiments of the invention, cells associated with the medical condition overexpress the GnRH receptor.

According to some embodiments of the invention, the medical condition is cancer.

According to some embodiments of the invention, the cancer is selected from the group consisting of breast cancer, prostate cancer, ovarian cancer, endometrial cancer, bladder cancer, lung cancer, melanoma, leukemia, glioblastoma and pancreatic cancer.

According to some embodiments of the invention, the cancer is pancreatic cancer.

According to an aspect of some embodiments of the present invention there is provided a nucleic acid construct comprising a nucleic acid sequence encoding a fusion protein comprising a DNA binding domain (DBD) of a transcription factor that binds a regulatory region of a gonadotropin gene attached to a chromatin modifying domain that represses transcription upon association with the DBD.

According to some embodiments of the invention, the transcription factor is selected from the group consisting of Sf-1, Egr-1 and Nur77.

According to some embodiments of the invention, the transcription factor is Sf-1.

According to some embodiments of the invention, the DBD of Sf-1 comprises SEQ ID NO: 2.

According to some embodiments of the invention, the DBD of Egr-1 comprises SEQ ID NO: 4

According to some embodiments of the invention, the chromatin modifying domain is a KRAB domain, catalytic domain of DNMT3a or HP1α.

According to some embodiments of the invention, the chromatin modifying domain is a KRAB domain.

According to some embodiments of the invention, the KRAB domain comprises SEQ ID NO: 7 or 8.

According to an aspect of some embodiments of the present invention there is provided a composition comprising the nucleic acid construct, attached to—or encapsulated by a cell penetrating moiety.

According to some embodiments of the invention, the penetrating moiety is a cell penetrating peptide.

According to an aspect of some embodiments of the present invention there is provided a composition comprising the nucleic acid construct attached to a gonadotrope-targeting moiety.

According to some embodiments of the invention, the composition comprises a gonadotrope-targeting moiety attached to the nucleic acid construct or the cell penetrating moiety.

According to some embodiments of the invention, the gonadotrope-targeting moiety is a GnRH receptor binding protein.

According to some embodiments of the invention, the gonadotrope-targeting moiety is attached to the nucleic acid construct or the composition using a linker.

According to some embodiments of the invention, the linker comprises a cleavage motif that can be cleaved by a proteolytic enzyme secreted from the pituitary.

According to some embodiments of the invention, the proteolytic enzyme is selected from the group consisting of PACE4, prohormone convertases (PC)1/3, PC2, PC4 PC5/6 and furin.

According to some embodiments of the invention, the proteolytic enzyme is selected from the group consisting of MMP, ADAM, ADAMTS, Kallikreins, PSA, uPA, Furin, PCSK, trypsin-like protease and thrombin.

According to some embodiments of the invention, the proteolytic enzyme is PACE4.

According to an aspect of some embodiments of the present invention there is provided a composition comprising a GnRH receptor binding protein attached to a cell penetrating peptide by a linker comprising a cleavage motif that can be cleaved by PACE4, wherein the cell penetrating peptide is attached to- or encapsulates a nucleic acid construct encoding a fusion protein comprising a DNA binding domain (DBD) of Sf-1 attached to a KRAB domain.

According to some embodiments of the invention, the GnRH receptor binding protein comprises human GnRH.

According to some embodiments of the invention, the GnRH receptor binding protein comprises SEQ ID NO: 9.

According to some embodiments of the invention, the therapeutic agent is a contraceptive.

According to some embodiments of the invention, the therapeutic agent regulates expression of a gonadotropin gene.

According to an aspect of some embodiments of the present invention there is provided a method of contraception in a mammal, the method comprising administering to the mammal an effective amount of the nucleic acid construct or the composition, thereby effecting contraception in the mammal.

According to an aspect of some embodiments of the present invention there is provided the nucleic acid construct or the composition, for use in contraception in a mammal.

According to some embodiments of the invention, the mammal is a non-human mammal.

According to an aspect of some embodiments of the present invention there is provided a method of decreasing or preventing sex-steroid driven behavior in a non-human mammal, the method comprising administering to the non-human mammal an effective amount of the nucleic acid construct or the composition, thereby decreasing or preventing the sex-steroid driven behavior in the non-human mammal.

According to some embodiments of the invention, the non-human mammal is a male.

According to some embodiments of the invention, the sex-steroid driven behavior is selected from the group consisting of sexual behavior towards bitches in heat, aggression, territoriality and urination to mark territory.

According to some embodiments of the invention, the non-human mammal is a dog, cat, pig, horse or deer.

According to some embodiments of the invention, the non-human mammal did not reach sexual maturation.

According to some embodiments of the invention, the non-human mammal is sexually mature.

According to an aspect of some embodiments of the present invention there is provided a method of treating a medical condition selected from the group consisting of steroid-dependent cancer, uterine infection, uterine fibroids and endometriosis in a subject in need thereof, the method comprising administering to the subject a therapeutically effective amount of the nucleic acid construct or the composition, thereby treating the medical condition in the subject.

According to an aspect of some embodiments of the present invention there is provided the nucleic acid construct or the composition, for use in treating a medical condition selected from the group consisting of steroid-dependent cancer, uterine infection, uterine fibroids and endometriosis in a subject in need thereof.

According to some embodiments of the invention, the cancer is selected from the group consisting of prostate cancer and breast cancer.

According to some embodiments of the invention, the subject is a human.

According to some embodiments of the invention, the subject no longer requires their fertility.

According to some embodiments of the invention, the nucleic acid construct or the composition is administered intranasally, subcutaneously or intramuscularly.

According to some embodiments of the invention, the nucleic acid construct or the composition is administered intranasally.

According to some embodiments of the invention, the nucleic acid construct or the composition is administered via a slow release implant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of the pituitary gland and its hormones in the central control of reproduction [adapted from Melamed P. Trends Endocrinol Metab. (2008) 19(1):25-31]. Luteinizing hormone (LH) and follicle stimulating hormone (FSH) are synthesized and released from the pituitary gland in response to stimulation by the hypothalamic GnRH. LH and FSH travel through the circulation to act on the ovaries and testes to produce mature germ cells and the sex steroids.

FIG. 2 shows schematic representations of constructs containing coding sequences of DNA binding domains (DBD) of Sf-1 and Egr-1 transcription factors ligated to the chromatin modifying KRAB domain. All constructs also contain the sequence encoding GFP at the 3′ end.

FIGS. 3A-C show the effects of the generated constructs (see FIG. 2) on expression of gonadotropin genes and Gnrhr. FIG. 3A shows mRNA expression levels of Cga and Lhb in immature partially-differentiated αT3-1 gonadotropes transiently transfected with the indicated constructs, as determined 2 weeks (8 duplications) following transfection in GFP-positive cells by qPCR analysis. Levels were normalized to those of Rplp0 and are shown (mean±SEM) relative to levels in control non-transfected cells. FIG. 3B shows mRNA expression levels of Cga, Lhb, Fshb and Gnrhr in the mature fully-differentiated gonadotrope LβT2 cell line, transiently transfected with the indicated constructs, as determined 12 days following transfection in GFP-positive cells by qPCR analysis. FIG. 3C shows additional results from a similar experiment repeated twice more (mean±SEM). Levels were normalized to those of Rplp0 and are shown relative to levels in control non-transfected cells.

FIGS. 4A-D demonstrate direct binding to the regulatory regions of the gonadotropin and Gnrhr genes. FIG. 4A is a schematic representation of sites reported previously to bind Sf-1 and Egr-1 in the promoter of each gene (namely Lhb, Cga and Gnrhr). Those bracketed mark putative binding sites based on sequence. FIG. 4B-D demonstrate ChIP analysis in αT3-1 cells expressing KRAB-Sf-1-GFP, KRAB-Egr-1-GFP or neither (WT non-transfected cells). ChIP was carried out with antibody to GFP to determine the association of the GFP-tagged proteins in regions upstream of Cga (FIG. 4B), Lhb (FIG. 4C) or Gnrhr (FIG. 4D) genes, with levels at Gapdh shown as negative control. All levels are presented relative to the levels of DNA at the same location in input samples and are shown as mean±SEM, n=3.

FIGS. 5A-E demonstrate the effects of the KRAB-Sf-1 construct on the chromatin of gonadotropin genes. Immature αT3-1 (FIG. 5A) and mature LβT2 (FIG. 5B) gonadotrope cell lines were stably transfected with the indicated construct. Following positive selection, ChTP was carried out for histone H3 using primers to various regions at the 5′ end of the Cga gene (FIG. 5A) and Lhb gene (FIG. 5B), and a satellite sequence or Atoh as control. Levels of DNA immunoprecipitated (IP) are shown relative to the levels in the input samples. FIGS. 5C and D show repeats of the experiment (n=3) in αT3-1 cells, with results shown relative to the mean of Atoh levels. FIG. 5E shows ChIP analysis carried out in LβT2 5 weeks following transient transfection with KRAB-Sf-1, as compared to control WT LβT2 non-transfected cells. Levels of H3 associated with Cga and Lhb genes were assessed and are presented as in FIGS. 5A-B with satellite DNA as negative control, and are shown as mean±SEM, n=3.

FIGS. 6A-B demonstrate that KRAB-Sf-1 induces H3K9me3 at both Cga and Lhb genes. ChTP analysis was carried out with an anti-H3K9me3 antibody in LβT2 cells transfected with KRAB-Sf-1, as compared to control WT LβT2 non-transfected cells, and levels at Cga (FIG. 6A) and Lhb (FIG. 6B) gene promoters were assessed. Level of the modified histone at each genomic locus is shown relative to that at the Atoh positive control gene.

FIGS. 7A-B demonstrate that KRAB-Sf-1 induces DNA methylation upstream of the Lhb gene. FIG. 7A is a schematic representation of the promoter and region upstream of the Lhb promoter rich in CpGs (short vertical lines), which are most concentrated in the region between −488 and −912 bp upstream from the TSS. FIG. 7B shows MeDIP analysis of the region shown in FIG. 7A carried out using an antibody to 5mC methylated DNA, with Gapdh as a negative control. Data were analyzed and are presented as in the ChTP experiments in FIGS. 6A-B.

FIGS. 8A-E demonstrate that a minimal delivery protein (MDP) packages DNA and binds the GnRHR but does not enter the cells. FIG. 8A is a schematic representation of a MDP containing protamine as a cell penetrating peptide (CPP), a NLS sequence and C-terminal cysteine cross-linked to GnRHLys6. FIG. 8B shows western blot analysis using an anti-GnRH antibody, confirming successful cross-linking of GnRH. FIG. 8C shows Egr-1 mRNA levels (to indicate activation of the GnRHR) following treatment of LβT2 cells with 100 nM GnRHLys6 or the MDP for 1 hour, as determined by qPCR. Results are shown as mean±SEM, n=3. FIG. 8D demonstrates an EtBr displacement assay performed using MBP at 0-400 ng, to indicate the degree of packaging (left graph), compared to that of protamine sulfate-packaged DNA, as a positive control (right graph). In FIG. 8E, LβT2 cells were treated with the MDP at two concentrations (200 ng DNA+300 ng MDP or 1 μg DNA+1.5 μg MDP), or 200 ng DNA was transfected using Polyjet reagent (positive control). Following 72 hours, RNA was extracted, purified and treated with DNAse, and qPCR performed using primers targeting the adaptor and GFP, and then by nested PCR targeting just the GFP. Results are shown as mean±SEM, n=3.

FIGS. 9A-B demonstrate that a CPP successfully delivers DNA into the cells, but not when cross-linked to GnRH, using Traut reagent and sulfo GMBS. FIG. 9A is a schematic representation of the CPP (NF51) cross-linked to GnRH using this approach and then packaged with the DNA. In FIG. 9B, LβT2 or MEF cells were treated with the NF51-DNA alone, or after its crosslinking to GnRH Lys6. PCR was carried out as in FIG. 8E. Also shown are mRNA levels in samples that lacked the reverse transcriptase (RT). The graph on the right shows mRNA levels relative to no RT indicating the transfection efficiency, after removing background noise from any remnants of the plasmid DNA.

FIGS. 10A-B demonstrate PACE4 cleavage activity in gonadotrope cells. Media was collected from LβT2 or MEF cells following 3 hours incubation, filtered and incubated with Boc-Arg-Val-Arg-Arg-MCA (SEQ ID NO: 96). Fluorescence was measured and relative (FIG. 10A), or absolute (FIG. 10B) readings shown as mean±SEM, n=3.

FIGS. 11A-E show schematic representations of a vehicle suggested by specific embodiments of the invention for targeted delivery of a polynucleotide to a GnRH receptor expressing cell: e.g. a KRAB-Sf-1 construct to gonadotrope cells. A CPP packaging DNA is conjugated to a GnRH analog via a short linker containing a cleavage motif for a proteolytic enzyme secreted by the cell e.g. PACE4 (e.g. peptide 2, SEQ ID NO: 102, FIG. 11A). The exemplary packaged DNA construct encodes a chimeric protein containing the DNA binding domain (DBD) of Sf-1 and the repressive KRAB domain (FIG. 11B). The GnRH carrier binds its receptor on the cell e.g. gonadotrope cells and the secreted proteolytic enzyme e.g. PACE4 (packmen) cleaves the CPP allowing it to transport the DNA construct into the cells (FIG. 11C). This DNA is transcribed to the exemplary chimeric protein, which binds Sf-1-targeted genes (Lhb, Fshb, Cga and Gnrhr) in the nucleus (FIG. 11D). The KRAB domain of the chimeric protein recruits other chromatin modifying enzymes (varied shapes) which close the chromatin and keep it repressed even after the chimeric protein is no longer present (FIG. 11E).

FIGS. 12A-B demonstrate delivery of a polynucleotide to GnRH receptor expressing cells. FIG. 12A shows activation of the GnRHR, inferred by induction of Egr1 mRNA levels (mean±SEM, n=3; as in FIG. 8C), by the Nf51 CPP with addition of a C-terminal cytosine (i.e. Peptide1, SEQ ID NO: 103 conjugated through Gly6 to the δ-NH2 group of ornithine 1 of SEQ ID NO: 101) crosslinked directly to GnRH Lys6 via GMBS, and by GnRH Lys6 similarly cross-linked to Peptide 2 (SEQ ID NO: 102) which includes a PACE4 cleavage site. FIG. 12B demonstrates specific delivery of a polynucleotide to GnRH receptor expressing cells, by the GnRH Lys6-Peptide 2. αT3-1 gonadotrope cells stably expressing GFP were mixed 1:10 with MEFs, and incubated for 1.5 hours with this GnRH delivery vehicle, loaded with a DNA encoding mCherry. Following 48 hours, cell were sorted by FACS to determine the numbers of cells expressing each fluorescent protein in a quantitative manner. The mCherry expression was detected in 58% of the αT3-1 gonadotrope cells, and in 3% of the MEFs.

FIG. 13 demonstrates selective delivery of a functional polynucleotide to GnRH receptor expressing cells. Murine αT3-1 gonadotrope cells were mixed ˜1:10 with human non-pituitary cells, and incubated for 1.5 hours with the GnRH delivery vehicle shown in FIG. 11A loaded with a DNA encoding KRAB-Sf1 or GFP (control). Following 13 days, cell were harvested, RNA extracted and Cga mRNA levels were determined by qPCR.

FIG. 14 demonstrates secretion of a functional PACE4 from pancreatic cells. Media was collected from cultured PANC-1 pancreatic cell line or HEK cells, filtered and incubated with a reporter peptide substrate (Boc-Arg-Val-Arg-Arg-MCA, SEQ ID NO: 96), that contains PACE4 cleave motif and emits a fluorescent signal upon cleavage. Fluorescence is shown relative to levels in medium from HEK cells; mean±SEM, n=3.

### DESCRIPTION OF SPECIFIC EMBODIMENTS OF THE INVENTION

The present invention, in some embodiments thereof, relates to compositions and methods for treating a medical condition.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Treatment of multiple diseases include administration of drugs that regulate gene expression, induce cytotoxic or apoptotic effects, enable production of a protein or a peptide and the like. While drug delivery takes a variety of forms, depending on the disease, the agent to be delivered and the administration route; targeted delivery and techniques enabling efficient transfer of a drug into a target cell are of main interest.

Whilst reducing specific embodiments of the present invention to practice, the present inventors have generated a chimeric protein for reducing expression of gonadotropin through chromatin compaction, as a novel method for contraception. Specifically, as is illustrated hereinunder and in the examples section, which follows, a number of chimeric DNA constructs containing sequences encoding the DNA binding domain (DBD) of various transcription factors (Egr-1, Sf-1), which recognize the gonadotropin genes Cga, Lhb and Fshb, fused N-terminal or C-terminal to the chromatin modifying KRAB domain were constructed (Example 1). Transfection of these constructs into a αT3-1 gonadotrope cell line leads to chromatin compaction and reduced expression of the gonadotropin genes and also of the Gnrhr gene. Following, the present inventors devised the use of a GnRH receptor binding protein (e.g. GnRH or an analog thereof) for targeted delivery of the nucleic acid constructs. As is shown in Example 2 of the examples section, which follows, the present inventors found out that while a GnRH analog cross-linked to a cell penetrating peptide (CPP) could target the DNA construct to the gonadotropes, it could not enter the cell, suggesting that detachment of the CPP from the GnRH analog might facilitate DNA delivery. Taking these observations into account, the present inventors altered the CPP sequence to allow more direct cross-linking to the GnRH analog, and added to the delivery vehicle a cleavage motif of an endogenous protease, PACE4, that is secreted at very high levels from pituitary cells. In this way, the GnRH receptor binding protein targets the CPP+DNA to the gonadotrope cell surface, where it binds the GnRH receptor. At the cell surface, the proteolytic enzyme secreted from the cell cleaves the CPP from the GnRH receptor binding protein, which allows the CPP to transport the DNA into the cells. Following, the inventors show that indeed the generated GnRH based-carrier delivers DNA efficiently and specifically to GnRH receptor-expressing cells (Example 3). While the constructs designed by the inventors target the reproductive axis, the specificity of the designed delivery vehicle which includes both the GnRH-based targeting moiety and the release of the CPP+DNA by a proteolytic enzyme secreted by the target cell (e.g. PACE) enables the use of such a delivery vehicle in the delivery of other therapeutic constructs to other target cells e.g. cancer cells (see e.g. Example 4 of the Examples section which follows).

Thus, according to an aspect of the present invention, there is provided a composition comprising a polypeptide comprising a GnRH receptor binding protein and a polynucleotide coding a therapeutic agent, wherein said polypeptide is in association with said polynucleotide via a linker comprising a cleavage motif that can be cleaved by a proteolytic enzyme secreted by a cell expressing said GnRH receptor.

The term “polypeptide” or “protein” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

Peptide bonds (—CO—NH—) within the peptide may be substituted, for example, by N-methylated amide bonds (—N(CH3)-CO—), ester bonds (—C(═O)—O—), ketomethylene bonds (—CO-CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptides of some embodiments of the invention may be synthesized by any techniques known to those skilled in the art of peptide synthesis, for example but not limited to recombinant DNA techniques or solid phase peptide synthesis. According to specific embodiments, the polypeptide is produced by attachment (e.g. covalent attachment e.g. via a peptide bond) of two distinct polypeptides (e.g. one comprising the GnRH receptor binding protein and the other comprising the cleavage motif that can be cleaved by the proteolytic enzyme and optionally a CPP). Such attachment can be effected by cross-linking the two polypeptides. Cross-linking can be effected by methods known in the art including e.g. sulfo GMBS (can be commercially obtained from e.g. Thermo-Fisher). According to specific embodiments, to facilitate direct cross-linking between the two polypeptides, one of the polypeptides e.g. the polypeptide comprising the cleavage motif comprises a terminal (e.g. c-terminal) cysteine.

The polypeptide of some embodiments of the invention comprises a GnRH receptor binding protein.

As used herein, the term “GnRH receptor” refers to the polypeptide encoded by the GNRHR gene (corresponding to human Gene ID 2798). According to specific embodiments, the GnRH receptor is the human GnRH receptor, such as provided in the following UniProt No. P30968. Orthologues of GnRH receptor are contemplated typically for therapeutic e.g. veterinary uses.

As used herein, the phrase “GnRH receptor binding protein” refers to an amino acid sequence having a binding affinity (e.g., below 10⁻⁴ M) to a GnRH receptor.

The GnRH receptor binding protein of some embodiments of the invention serves as the targeting moiety for delivery of the polynucleotide of some embodiments of the invention into a cell expressing a GnRH receptor.

Assays for testing binding are well known in the art and include, but not limited to flow cytometry, ELISA, bio-layer interferometry Blitz® assay, HPLC, surface plasmon resonance (e.g. Biacore).

According to specific embodiments, the GnRH receptor binding protein comprises GnRH.

As used herein, the term “GnRH” (also known as gonadotropin-releasing hormone) refers to the polypeptide encoded by the GNRH1 gene (corresponding to human Gene ID 2796). According to specific embodiments, the GnRH is the human GnRH, such as provided in the following Accession Nos. NP_000816, NP_001076580 (SEQ ID NOs: 10-11). Orthologues of GnRH are contemplated typically for therapeutic e.g. veterinary uses.

The term “GnRH” as used herein includes full length GnRH or a fragment thereof or a homolog thereof which maintains at least the capability of the full length GnRH to specifically bind the GnRH receptor.

According to specific embodiments, the amino acid sequence of GnRH comprises SEQ ID NO: 88.

The homolog (naturally occurring or synthetically/recombinantly produced) can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide provided in SEQ ID NO: 10, 11 or 88 or a functional fragment thereof which exhibits the desired activity (i.e. at least binding a GnRH receptor).

Sequence identity or homology can be determined using any protein or nucleic acid sequence alignment algorithm such as Blast, ClustalW, and MUSCLE.

The homolog may also refer to an ortholog, a deletion, insertion, or substitution variant, including a conservative and non-conservative amino acid substitution.

According to specific embodiments, the GnRH receptor binding protein comprises a GnRH analog.

The analog may or may not have sequence similarity to GnRH as long as it is capable of least specifically binding the GnRH receptor.

Non-limiting examples of GnRH analogs that can be used with specific embodiments of the present invention include, buserelin, gonadorelin, goserelin, histrelin, leuprorelin, nafarelin, deslorelin and fertirelin.

According to specific embodiments, the GnRH analog is GnRH-Lys6. Thus, according to specific embodiments, the GnRH receptor binding protein comprises SEQ ID NO: 9.

According to specific embodiments, the GnRH receptor binding protein consists of SEQ ID NO: 9.

The polypeptide of some embodiments of the invention is in association with a polynucleotide encoding a therapeutic agent.

The therapeutic agent of some embodiments of the invention may act at the genomic level (i.e., activation of transcription via promoters, enhancers, regulatory elements), at the transcript level (i.e., correct splicing, polyadenylation, activation of translation, chromatin modification) or at the protein level (i.e., post-translational modifications, interaction with substrates and the like).

Hence, according to specific embodiments, the therapeutic agent acts as a polynucleotide.

According to other specific embodiments, the therapeutic agent acts as the polypeptide encoded by the polynucleotide.

Non-limiting Examples of therapeutic agents include an RNA silencing agent, a genome editing agent, a chromatin modifying agent, a transcription factor, a cytotoxic agent, an apoptotic agent, a toxin, a contraceptive etc.

According to specific embodiments, the therapeutic agent is a contraceptive.

According to specific embodiments, the therapeutic agent regulates an expression of a gonadotropin gene. Additional description on gonadotropin genes is provided hereinbelow.

As used herein the term “polynucleotide” refers to a single or double stranded nucleic acid sequence in the form of an RNA sequence (e.g. mRNA), a complementary polynucleotide sequence (cDNA), a genomic polynucleotide sequence (e.g. sequence isolated from a chromosome), a composite polynucleotide sequences (e.g., a combination of the above) or mimetic or analog thereof. This term includes polynucleotides derived from naturally occurring nucleic acids molecules (e.g., RNA or DNA), synthetic polynucleotide and/or oligonucleotide molecules composed of naturally occurring bases, sugars, and covalent internucleoside linkages (e.g., backbone), as well as synthetic polynucleotides having non-naturally occurring portions, which function similarly to the respective naturally occurring portions.

Polynucleotides designed according to the teachings of some embodiments of the invention can be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

According to specific embodiments, the polynucleotide is comprised in a nucleic acid construct suitable for expression in a cell e.g. a mammalian cell. Such a nucleic acid construct includes a promoter sequence for directing transcription of the polynucleotide sequence in the cell in a constitutive or inducible manner.

According to specific embodiments, the promoter is heterologous to the therapeutic polynucleotide.

Non-limiting examples of constitutive promoters suitable for use with some embodiments of the invention are promoter sequences which are active under most environmental conditions and most types of cells such as the cytomegalovirus (CMV) and Rous sarcoma virus (RSV). Inducible mammalian promoters that can be used with specific embodiments of the invention are known to those of skill in the art (see, e.g. Bitter et al. (1987) Methods in Enzymology 153: 516-544) and include for example the tetracycline-inducible promoter (Zabala M, et al., Cancer Res. 2004, 64(8): 2799-804).

As used herein, the phrase “in association with” refers to direct or indirect binding (or attachment) of the polypeptide comprising the GnRH receptor binding protein to the polynucleotide coding the therapeutic agent or to a particle comprising the polynucleotide coding a therapeutic agent.

According to specific embodiments, the polypeptide is in association with the polynucleotide via a linker comprising a cleavage motif that can be cleaved by a proteolytic enzyme secreted by a cell expressing GnRH receptor. Typically, such a linker is a polypeptide linker.

As used herein, the term “proteolytic enzyme”, also known as “protease” refers to an enzyme that catalyzes the breakdown of an amino acid bond by hydrolysis at a specific cleavage motif, and is secreted by a cell. Numerous such proteolytic enzymes are known in the art. Non-limiting examples include MMP, ADAM, ADAMTS, Kallikreins, PSA, uPA, Furin, PCSK, trypsin-like protease and thrombin.

According to specific embodiments, the proteolytic enzyme is secreted from the pituitary.

According to specific embodiments, the proteolytic enzyme is selected from the group consisting of PACE4, prohormone convertases (PC)1/3, PC2, PC4 PC5/6 and furin.

According to specific embodiments, the proteolytic enzyme is PACE4.

As used herein, the term “PACE4”, also known as “Proprotein convertase subtilisin/kexin 6”, refers to a proteolytic enzyme encoded by the PCSK6 gene (corresponding to human Gene ID 5046). According to specific embodiments, the PACE4 is the human PACE4, such as provided in the following UniProt No. P29122. Orthologues of PACE4 are contemplated typically for therapeutic e.g. veterinary uses.

The cleavage motif of proteolytic enzymes that can be used with specific embodiments of the invention, are well known to the skilled in the art.

Thus, for example, the cleavage motif of PACE4 comprises RVRR (SEQ ID NO: 97), the cleavage motif of thrombin comprises PRGW (SEQ ID NO: 98); the cleavage motif of uPA comprises GGSGRSANAKC (SEQ ID NO: 99); trypsin cleaves C-terminal to R and K; PSA cleaves its substrates preferentially after Y, Q and less commonly after L.

According to specific embodiments the linker comprises a terminal (e.g. c-terminal) cysteine.

Thus, according to specific embodiments, the polypeptide comprising the GnRH receptor binding protein is attached to the linker comprising the cleavage motif via a cysteine.

Methods of binding a polynucleotide to a polypeptide are known in the art and include for example covalent, non-covalent, hydrogen, chemical and/or electrostatic bonds.

According to specific embodiments, the polynucleotide is attached to- or encapsulated by—a cell penetrating moiety.

As used herein the phrase “cell penetrating moiety” refers to a moiety which enhances translocation of the polynucleotide coding the therapeutic agent across a cell membrane. Non-limiting examples of cell penetrating moieties include cell penetrating peptides and lipid particles.

For example, the polynucleotide may be incorporated into a particulated delivery vehicle, e.g., a liposome, or a nano- or microparticle, by any of the methods known in the art [e.g. Liposome Technology, Vol. II, Incorporation of Drugs, Proteins, and Genetic Material, CRC Press; Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43; Ramishetti et al. Adv Mater. 2020 Jan. 30:e1906128, International Patent Application publication Nos. WO2018/015881 and WO2018087753, WO2017194454 and US Patent Application Publication no. US20130245107, the contents of which are fully incorporated herein by reference].

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. Liposomes can be of different sizes, may contain a low or a high pH and may be of different charge.

Method of binding a particle to a polypeptide are known in the art and are also disclosed in e.g. International Patent Application Publication NO. WO2018/015881, U.S. Pat. Nos. 5,171,578, 5,204,096 and 5,258,499, the contents of which are fully incorporated herein by reference.

According to specific embodiments, the cell penetrating moiety is a cell penetrating peptide.

As used herein, a “cell-penetrating peptide (CPP)” is a peptide that comprises a short (about 12-40 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention may include, but are not limited to, protamine, NF51, penetratin, transportan, pIsl, TAT(48-60), pVEC, MTS, and MAP. According to specific embodiments, the CPP is NF51 such as provided e.g. in SEQ ID NO: 103 conjugated through Gly6 to the δ-NH2 group of ornithine 1 of SEQ ID NO: 100. According to specific embodiments, the CPP comprises a terminal cysteine (e.g., SEQ ID NO: 101). Such a cysteine may facilitate direct cross-linking between the polypeptide and the CPP using e.g. sulfo-GMBS. Protocols for producing CPPs-cargos conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633]. An example of such a protocol is also described in the Examples section, which follows, which serves as an integral part of the specification as filed.

According to specific embodiments, the linker comprising the cleavage motif that can be cleaved by the proteolytic enzyme is located between the polypeptide comprising the GnRH receptor binding protein and the cell penetrating moiety, such that cleavage by the proteolytic enzyme releases the polynucleotide coding the therapeutic agent attached to- or encapsulated by the cell penetrating moiety from the polypeptide.

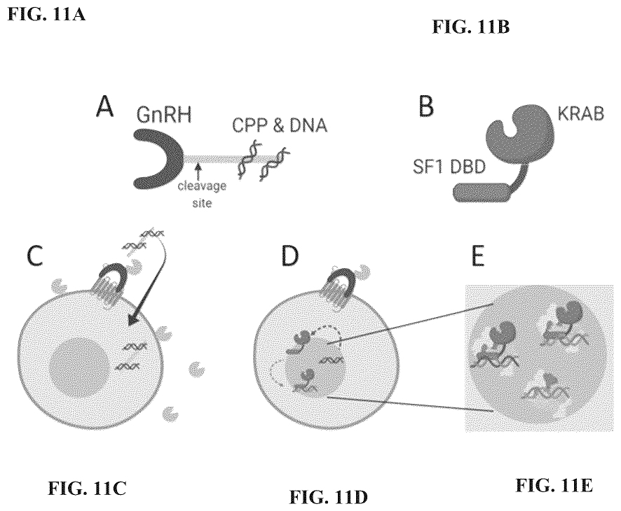

Thus, according to an aspect of the present invention, there is provided a composition comprising a GnRH receptor binding protein attached to a cell penetrating peptide by a linker comprising a cleavage motif that can be cleaved by a secreted proteolytic enzyme e.g. (PACE4), wherein the cell penetrating peptide is attached to- or encapsulates a polynucleotide coding a therapeutic agent. A non-limiting schematic representation of such a composition is provided in FIG. 11A.

According to an additional or an alternative aspect of the present invention there is provided a composition comprising a GnRH receptor binding protein attached to a cell penetrating peptide by a linker comprising a cleavage motif that can be cleaved by PACE4, wherein said cell penetrating peptide is attached to- or encapsulates a nucleic acid construct encoding a fusion protein comprising a DNA binding domain (DBD) of Sf-1 attached to a KRAB domain, as further described infra.

According to specific embodiments, the linker comprising the cleavage motif comprises a terminal cysteine.

According to specific embodiments, the polynucleotide or the cell penetrating moiety is attached (directly or indirectly) to a localization signal that can direct the polynucleotide to a particular intracellular compartment e.g. nuclear, ER, mitochondrial, cytoplasmic. Such localization signals are well known to the skilled in the art.

According to specific embodiments, the localization sequence is a nuclear localization sequence (NLS).

Specific embodiments of the invention also contemplate the use of the compositions disclosed herein for treating a medical condition, wherein cells associated with the medical condition express GnRH receptor and secrete the proteolytic enzyme.

Thus, according to an aspect of the present invention, there is provided a method of treating a medical condition in a subject in need thereof, the method comprising administering to the subject a therapeutically effective amount of the composition of disclosed herein, wherein cells associated with said medical condition express said GnRH receptor and secrete said proteolytic enzyme, thereby treating the medical condition in the subject.

According to an additional or an alternative aspect of the present invention, there is provided the composition disclosed herein, for use in treating a medical condition in a subject in need thereof, wherein cells associated with said medical condition express said GnRH receptor and secrete said proteolytic enzyme.

Methods of determining expression are well known in the art and include e.g. western blot, flow cytometry, ELISA.

According to specific embodiments, the cells associated with the medical condition express the GnRH receptor above a pre-determined threshold.

According to specific embodiments, the cells associated with the medical condition overexpress the GnRH receptor.

As used herein, the term “overexpress” refers to an increased expression of a GnRH receptor as compared to a healthy cell.

According to specific embodiments, the healthy cell is of the same type as the diseased cell.

According to specific embodiments, the increased expression is at least 1.5 fold, at least 2 fold, at least 3 fold, at least 5 fold, at least 10 fold, or at least 20 fold as compared the level of GnRH receptor in a healthy cell as measured using the same assay such as e.g. western blot, flow cytometry, ELISA.

Methods of determining cell secretion of a proteolytic enzyme are known in the art, and include ELISA or an activity assay such as hydrolysis of a substrate comprising the proteolytic enzyme cleavage motif, as further described in the Examples section which follows.

According to specific embodiments, the cells associated with the medical condition secrete the proteolytic enzyme above a pre-determined threshold.

According to specific embodiments, the cells associated with the medical condition secrete higher amounts of the proteolytic enzyme as compared to healthy cells.

According to specific embodiments, the higher secretion is at least 1.5 fold, at least 2 fold, at least 3 fold, at least 5 fold, at least 10 fold, or at least 20 fold as compared the amount of the proteolytic enzyme secreted by a healthy cell as measured using the same assay such as ELISA or an activity assay (e.g. hydrolysis of a substrate).

According to specific embodiments, the medical condition is cancer.

Non-limiting examples of cancers that can be treated according to specific embodiments of the invention include breast cancer, prostate cancer, ovarian cancer, endometrial cancer, bladder cancer, lung cancer, melanoma, leukemia, glioblastoma, pancreatic cancer and steroid-dependent cancer.

According to specific embodiments, the cancer is pancreatic cancer.

As the present inventors have generated a chimeric protein for reducing expression of gonadotropin, specific embodiments of the invention also contemplate the use of the compositions disclosed herein as a contraception, for of decreasing or preventing or for treating sex-steroid driven behavior, steroid-dependent cancer, uterine infection, uterine fibroids and/or endometriosis.

Thus, according to an additional or an alternative aspect of the present invention there is provided a method of contraception in a mammal, the method comprising administering to the mammal an effective amount of the composition disclosed herein, thereby effecting contraception in the mammal.

According to an additional or an alternative aspect of the present invention there is provided the composition disclosed herein, for use in contraception in a mammal.

According to an additional or an alternative aspect of the present invention there is provided a method of decreasing or preventing sex-steroid driven behavior in a non-human mammal, the method comprising administering to the non-human mammal an effective amount of the composition disclosed herein, thereby decreasing or preventing the sex-steroid driven behavior in the non-human mammal.

According to specific embodiments, the sex-steroid driven behavior is selected from the group consisting of sexual behavior towards bitches in heat, aggression, territoriality and urination to mark territory.

According to an additional or an alternative aspect of the present invention there is provided a method of treating a medical condition selected from the group consisting of steroid-dependent cancer, uterine infection, uterine fibroids and endometriosis in a subject in need thereof, the method comprising administering to the subject a therapeutically effective amount of the composition disclosed herein, thereby treating the medical condition in the subject.

According to an additional or an alternative aspect of the present invention there is provided the composition disclosed herein, for use in treating a medical condition selected from the group consisting of steroid-dependent cancer, uterine infection, uterine fibroids and endometriosis in a subject in need thereof.

According to specific embodiments, the cancer is selected from the group consisting of prostate cancer and breast cancer.

As used herein, the term “subject” includes mammals, at any age and of any gender which suffer from the pathology. According to specific embodiments, this term encompasses individuals who are at risk to develop the pathology.

According to specific embodiments, cells of the subject (e.g. gonadotrope cells) express a GnRH receptor.

According to specific embodiments, the subject is a human.

According to other specific embodiments, the subject is a non-human mammal. Non-limiting examples of non-human mammals include dog, cat, pig, horse, deer and the like.

According to specific embodiments, the subject is a male.

According to other specific embodiments, the subject is a female.

According to specific embodiments, the subject did not reach sexual maturation.

According to other specific embodiments, the subject is sexually mature.

Methods of determining sexual maturation are known in the art and include, but not limited to hormone assessment, the James M. Tanner Sexual Maturity Rating Scale (SMR) and the pubertal development scale (PDS).

According to specific embodiments, the subject no longer requires their fertility.

In addition, as the present inventors have generated a chimeric protein for reducing expression of gonadotropin through chromatin compaction, specific embodiments of the present invention also contemplates such constructs and their further therapeutic use.

Thus, according to an aspect of the present invention, there is provided a nucleic acid construct comprising a nucleic acid sequence encoding a fusion protein comprising a DNA binding domain (DBD) of a transcription factor that binds a regulatory region of a gonadotropin gene; attached to a chromatin modifying domain that represses transcription upon association with (i.e. binding of) said DBD.

The term “DNA binding domain (DBD)” refers to an amino acid sequence that is able to bind to DNA. Non-limiting examples of DBDs include the zinc finger, leucine zipper, helix-turn-helix, helix-loop-helix, HMG-box, Wor3 domain, OB-fold domain, and others. Methods of determining binding to DNA are well known in the art and include, but not limited to electrophoretic mobility-shift assay (EMSA), DNase I footprinting and chromatin immunoprecipitation (ChIP).

According to specific embodiments, the DBD of the transcription factor comprised in the fusion protein does not comprise the full length native transcription factor.

The transcription factor of some embodiments of the invention binds and regulates transcription of a gonadotropin gene.

As used herein, the term “gonadotropin gene” refers to a gene encoding a subunit of the gonadotropin hormones follicle-stimulating hormone (FSH) and/or luteinizing hormone (LH), each of which are composed of two subunits: alpha and beta, wherein the alpha subunit in FSH and LH is identical and the beta subunit is distinct.

Thus according to specific embodiments, the gonadotropin gene is CGA (corresponding to human Gene ID 1081) encoding the alpha subunit of the gonadotropin hormones.

Orthologues of any of the components of the compositions disclosed herein, such as the gonadotropins, are contemplated typically for therapeutic e.g. veterinary uses.

According to specific embodiments, the gonadotropin gene is FSHB (corresponding to human Gene ID 2488) encoding the FSH, beta polypeptide.

According to specific embodiments, the gonadotropin gene is LHB (corresponding to human Gene ID 3972) encoding the LH, beta polypeptide.

According to specific embodiments, the transcription factor regulates transcription of at least 1, at least two or all the gonadotropin genes.

Non-limiting examples of transcription factors of a gonadotropin gene include SF-1, Egr-1 and Nur77.

As used herein, the term “SF-1” (also known as steroidogenic factor 1) refers to the polypeptide of the NR5A1 gene (corresponding to human Gene ID 2516). According to specific embodiments, the SF-1 is the mouse SF-1, such as provided in the following Accession Nos. NP_001303616, NP_620639. According to specific embodiments, the SF-1 is the human SF-1, such as provided in the following Accession No. NP_004950.

According to specific embodiments, the DBD of Sf-1 comprises SEQ ID NO: 1.

According to specific embodiments, the nucleic acid sequence encoding the DBD of Sf-1 comprises SEQ ID NO: 2

The term “DBD of Sf-1” also encompasses functional fragments and/or homologues (naturally occurring or synthetically/recombinantly produced), which maintain the capability of binding Sf-1 target genes. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 1; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same e.g. SEQ ID NO: 2.

As used herein, the term “Egr-1” (also known as early growth response protein 1, Zif268 and zinc finger protein 225) refers to the polypeptide of the EGR1 gene (corresponding to human Gene ID 1958). According to specific embodiments, the Egr-1 is the mouse Egr-1, such as provided in the following Accession No. NP_031939. According to specific embodiments, the Egr-1 is the human Egr-1, such as provided in the following Accession No. NP_001955.

According to specific embodiments, the DBD of Egr-1 comprises SEQ ID NO: 3.

According to specific embodiments, the nucleic acid sequence encoding the DBD of Egr-1 comprises SEQ ID NO: 4.

The term “DBD of Egr-1” also encompasses functional fragments and/or homologues (naturally occurring or synthetically/recombinantly produced), which maintain the capability of binding Egr-1 target genes. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 3; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same e.g. SEQ ID NO: 4.

As used herein, the term “Nur77” (also known as nuclear receptor 4A1, TR3, and NGFI-B) refers to the polypeptide of the NR4A1 gene (corresponding to human Gene ID 3164). According to specific embodiments, the Nur77 is the mouse Nur77, such as provided in the following Accession No. NP_034574. According to specific embodiments, the Nur77 is the human Nur77, such as provided in the following Accession No. NP_001189162, NP_001189163, NP_002126, NP_775180, NP_002126.

According to specific embodiments, the DBD of Nur77 comprises SEQ ID NO: 90.

According to specific embodiments, the nucleic acid sequence encoding the DBD of Nur77 comprises SEQ ID NO: 91.

The term “DBD of Nur77” also encompasses functional fragments and/or homologues (naturally occurring or synthetically/recombinantly produced), which maintain the capability of binding Nur77 target genes. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 90; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same e.g. SEQ ID NO: 91.

As used herein, the phrase “chromatin modifying domain” refers to an amino acid sequence which upon indirectly binding to DNA via a targeting moiety (e.g. DBD) recruits epigenetic machinery (e.g. chromatin, histone or DNA modifying enzymes) to close the chromatin packaging, leading to repression of transcription of a gene to which it is targeted (i.e. via the associated DBD). Non-limiting examples of chromatin modifying domains that can be used with specific embodiments of the invention include KRAB domain (also known as Krüppel associated box domain, InterPro database entry IPR001909, Pfam database entry PF0135); functional domains of DNA methyl transferases, DNMT1, DNMT3A, DNMT3B or associated protein DNMT3L; catalytic domain of any of the histone deacetylates (HDACs), HP1α or repressive methyl transferases.

According to specific embodiments, the chromatin modifying domain is a KRAB domain.

Non-limiting examples of KRAB domains that can be used with specific embodiments of the present invention comprise SEQ ID NOs: 5 and 6.

According to specific embodiments, the nucleic acid sequence encoding the KRAB domain comprises SEQ ID NOs: 7 or 8.

According to specific embodiments, the chromatin modifying domain is HP1α.

According to specific embodiments, the HP1α comprises SEQ ID NO: 92.

According to specific embodiments, the nucleic acid sequence encoding the HP1α comprises SEQ ID NO: 93.

According to specific embodiments, the chromatin modifying domain is a catalytic domain of DNMT3a.

According to specific embodiments, the DNMT3a comprises SEQ ID NO: 94.

According to specific embodiments, the nucleic acid sequence encoding the DNMT3a comprises SEQ ID NO: 95.

According to specific embodiments, the nucleic acid construct is attached to- or encapsulated by a cell penetrating moiety.

A detailed description on cell penetrating moieties that can be used with specific embodiments of the invention is provided hereinabove.

According to specific embodiments, the nucleic acid construct is attached to a gonadotrope-targeting moiety.

As used herein, the phrase “gonadotrope targeting moiety” relates to a functional group which serves to target or direct the nucleic acid construct, the fusion polypeptide or the composition comprising same of some embodiments of the invention specifically to gonadotrope cells. Such targeting moieties include, but are not limited to hormones (e.g. GnRH or an analog thereof), antibodies, cell surface receptors, ligands, agonists, lipids, sugars and dextrans.

According to specific embodiments, the targeting moiety is a GnRH receptor binding protein. A detailed description of GnRH receptor binding proteins that can be used with specific embodiments of the invention is further provided hereinabove.

The attachment of the gonadotrope-targeting moiety to the nucleic acid construct may be direct or indirect.

Thus, according to specific embodiments, the gonadotrope-targeting moiety is attached to the cell penetrating moiety.

According to specific embodiments, the gonadotrope-targeting moiety is attached to the nucleic acid construct or the cell penetrating moiety via a linker.

Any linker known in the art can be used with specific embodiments of the invention.

According to a specific embodiment, the linker comprises a cleavage motif that can be cleaved by a proteolytic enzyme e.g. a proteolytic enzyme secreted by the pituitary.

Specific embodiments of the invention also contemplate the fusion protein encoded by the nucleic acid construct disclosed herein and compositions comprising same.

Thus, according to an aspect of the present invention, there is provided a fusion protein comprising a DNA binding domain (DBD) of a transcription factor that binds a regulatory region of a gonadotropin gene; attached to a chromatin modifying domain that represses transcription upon association with (i.e. binding of) said DBD.

According to an additional or alternative aspect of the present invention, there is provided a composition comprising the fusion polypeptide, a cell penetrating moiety and/or a gonadotrope-targeting moiety.

The composition of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to any of the components of the composition accountable for the biological effect.

Herein, the “components of the composition” refers to each of the elements and the combined composition as described herein including the polypeptide comprising a GnRH receptor binding protein, the polynucleotide coding the therapeutic agent, the linker comprising the cleavage motif that can be cleaved by the proteolytic enzyme and the cell penetrating moiety as disclosed herein.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

According to specific embodiments, the composition is administered intranasally, subcutaneously or intramuscularly.

According to specific embodiments, the composition is administered intranasally.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to specific embodiments, the composition is administered via a slow release implant.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a medical condition, substantially ameliorating clinical or aesthetical symptoms of a condition or substantially preventing the appearance of clinical or aesthetical symptoms of a condition. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

When reference is made to particular sequence listings, such reference is to be understood to encompass also sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Constructs—The DNA constructs were created by amplifying the relevant sequences as follows: SF1 and EGR1 sequences were amplified from expression plasmids; mKRAB and hKRAB were amplified from reverse transcribed RNA extracted from αT3-1 or HEK293 cells, respectively, using primers shown in Table 1 hereinbelow. These fragments were inserted into pGem-T easy plasmid vector (Promega), and sequences verified before continuing with the cloning. The mCherry (pmCherry-C1) and GFP (pEGFP-N1) plasmids were purchased from Clontech.

TABLE 1
PCR primers for cloning DNA fragments
(underline marks restriction sites)

| | | SEQ ID |
|---|---|---|
| | | NO: |
| SF1 F | AAGCTTATGGACTATTCGTACGACGA | 12 |
| SF1 R | GGTACCCCTGCTTTCTTCTGCTGCTTCA | 13 |
| EGR1 F | AAGCTTATGTATGCTTGCCCTGTCGAGT | 14 |
| EGR1 R | GGTACCCCGTCCTTCTGTCTTAAATGGAT | 15 |
| mKRAB F | CTCGAGATGGCCCCGAGACCTCCG | 16 |
| mKRAB R | AAGCTTGGTCTTCCAGTCTGGACAGA | 17 |
| hKRAB F | CTCGAGATGGATGCTAAGTCACTAAC | 18 |
| hKRAB R | AAGCTTTGCAGTCTCTGAATCAGGAT | 19 |

Cloning strategy—The mKRAB in pGem-T easy or hKRAB in pGem-T easy was cut with Xho1/HindIII and ligated to pEGF-N1 plasmid (Clontech) that includes the GFP sequence at the N terminal, which had been cut with Xho1/HindIII. This ligation product was then cut with Acc651/HindIII as vector and was ligated with SF1 or Egr1 in pGem-T easy, cut with Acc651/HindIII as insert.

Cell culture—The gonadotrope cell lines (αT3-1 and LβT2; as in references 5-6) were cultured as follows: αT3-1 cells in MEM alpha with 10% FCS, 1% pen strep, 1% L-glutamine, 1% sodium pyruvate, HEPES 1% (1M stock), 1% non-essential amino acids; LβT2 cells in DMEM high glucose with 10% FCS, 1% pen strep, 1% L-glutamine, 1% sodium pyruvate, 1% sodium bicarbonate. As appropriate the cells were transfected with Polyjet transfection reagent (SignaGen) at a ratio of 3 μl reagent to 1 μg DNA for 2×10⁶ cells. For stable transfections, the plasmids were linearized with Pcil and cleaned through Zymo purification kit (Zymo) which was transfected similarly.

RT-PCR—Real-time quantitative PCR was performed following RNA extraction (Trizol, Invitrogen followed by Zymo RNA purification kit+DNase) and reverse transcriptase (qScript cDNA synthesis kit, Quanta) using polymerase (2×qPCRBIO Fast qPCR SyGreen Blue Mix, Hi-ROX, PCR Biosystems) and the primers shown in Table 2 hereinbelow. Values were calculated using DNA standard curves and normalized to levels of Rplp0.

TABLE 2
PCR primers for expression analysis

| | | | SEQ ID |
|---|---|---|---|
| gene | name | sequence | NO: |
| Cga | aGSU 97F | ATGGATTACTACAGAAAATATGCAG | 20 |
| Cga | aGSU 196R | CCTGAATAATAAAGTCTCCATCAGG | 21 |
| Lhb | LHb CDS 179F | CTAGCATGGTCCGAGTACTG | 22 |
| Lhb | LHb CDS 301R | CTGAGGGCTACAGGAAAGGA | 23 |
| Fshb | FSH 637F | GCTGGAGAGCAATCTGCTGCCA | 24 |
| Fshb | FSH 795R | TATTGGGCCGAGCTGGGTCCTTA | 25 |
| Gnrhr | GnRHR +611F | CCACAGTCTTCTCGCAATGT | 26 |
| Gnrhr | GnRHR +891R | ACCAAATGCCTAGGACATAG | 27 |
| Rplp0 | mRPLP0 140F | GCGACCTGGAAGTCCAACTA | 28 |
| Rplp0 | hRPLP0 240R | ATCTGCTTGGAGCCCACAT | 29 |
| Egr1 | Egr1 554 F | AACAACCCTACGAGCACCTG | 30 |
| Egr1 | Egr1 683 R | AGCGGCCAGTATAGGTGATG | 31 |

Chromatin immunoprecipitation and methylated DNA immunoprecipitation—Chromatin immunoprecipitation (ChIP) was performed after 1% formaldehyde cross linking for 10 minutes at room temperature, and sonication of the DNA (for 10 cycles 15 seconds on, 10 seconds off for GFP ChTP and 60 cycles 15 seconds on, 10 seconds off for H3 and H3K9me3 ChIP), with antibody to GFP (AB290 Abcam), H3 (AB1791 Abcam), H3K9me3 (AB8898 Abcam) at 3 μg each per 10⁷ cells. Analysis of precipitated and input DNA was by qPCR, as above, using primers shown in Table 3 hereinbelow.

TABLE 3
PCR primers for ChIP analysis

| | | | SEQ |
|---|---|---|---|
| | | | ID |
| gene | name | sequence | NO: |
| GAPDH | GAPDH −226FP | GGAAGCAGCATTCAGGTCTC | 32 |
| GAPDH | GAPDH −148RP | CAGGATAGGACTCAGGGAATACAG | 33 |
| ATOH | Atoh1 −355F | CCCTCACTCAGGTCGCCTG | 34 |
| ATOH | Atoh1 −148R | CGTGCGAGGAGCCAATCA | 35 |
| satel- | major | GACGACTTGAAAAATGACGAAATC | 36 |
| lite | satellite F | | |
| satel- | major | CATATTCCAGGTCCTTCAGTGTGC | 37 |
| lite | satellite R | | |
| Cga | mGSU (−969)F | TATATGTACACATCTGTGGGT | 38 |
| Cga | mGSU (−820)R | GGTTAAGAGCATGAACTGCTC | 39 |
| Cga | mGSU −525F | CACACCTGGACATATCTACTGT | 40 |
| Cga | mGSU −425R | CAATTGCTCCAATTTCTTAAATTAAC | 41 |
| Cga | aGSU (−239)F | GCCAAATGCTCTCTCTTCATAAGC | 42 |
| Cga | aGSU (+84)R | CCTCTGGACTTTTTGAGGGATG | 43 |
| Cga | mGSU (−119)F | CCAAGTGCCATCCAATCACT | 44 |
| Cga | mGSU (−30)R | CCAGCAGAGTAATACAAATTGC | 45 |
| Cga | mGSU +47F | GTGGTCACAAATATTTTACTCTTT | 46 |
| Cga | mGSU +147R | ATCACCTGCCCAGAACAC | 47 |
| Cga | mGSU (−813)F | AGCCATCTCTCTAGACCCTA | 48 |
| Cga | mGSU (−572)R | GCAGCCTAAGAATTACTTAAGC | 49 |
| Cga | mGSU −429F | GGAGCAATTGTTTTATTTTTCTGT | 50 |
| Cga | mGSU −329R | ATTAGCTAAGTACCTGATATTTTCA | 51 |
| Cga | mGSU −334F | GCTAATTAAATGTGCTACTCTTAG | 52 |
| Cga | mGSU −235R | GGCCATTTTCTATGTAATATTATTG | 53 |
| Cga | mGSU −238F | GGCCAAATGCTCTCTCTTCA | 54 |
| Cga | mGSU −139R | TTGATCATATCACATTGCAACCC | 55 |
| Cga | mGSU −144F | GATCAATTGATGTCATGGTAATT | 56 |
| Cga | mGSU −45R | AATTGCATGTTCCCAGCTG | 57 |
| Cga | mGSU −50F | GCAATTTGTATTACTCTGCTGGT | 58 |
| Cga | mGSU +50R | GATTGTCGATCTGAGGGGAA | 59 |
| LHβ | LHB −518F | GACACACCCCTTACTTCCAGAG | 60 |
| LHβ | LH −419R | GGATGCCTGGATTGGGTCCAA | 61 |
| LHβ | LH−422F | ATCCTGATTAGGGGCTGGGAG | 62 |
| LHβ | LH −323R | GGCACAGCTTCAACCTTAGGTT | 63 |
| LHβ | LH −233F | CTCCCCTTTACCTTGTTTCCCGT | 64 |
| LHβ | LH −134R | CAGGGACTAGCTCCAGTGTC | 65 |
| LHβ | LH −139F | TCCCTGGCTTCCCTGACCTTG | 66 |
| LHβ | LH −40R | TGGGGGTGGCAAGGCCACTA | 67 |
| LHβ | LH −42F | CCACAACCCGCAGGTATAA | 68 |
| LHβ | LH +58R | GATTGGGAAGTACCCTGGG | 69 |
| LHβ | LH +55F | AATCCCACCAAACCCATAGATG | 70 |
| LHβ | LH +154R | GTTTATCTCCTGTTTGAAGTCCAC | 71 |
| LHβ | LH−395F | GCACCACCTCTGGTTGGATTGA | 72 |
| LHβ | LH−151R | GTGTCCCAGTGAATTGGCCTCA | 73 |
| LHβ | LHb (−269)F | CAATCTGGGGGTTCAGCGAG | 74 |
| LHβ | LHb (−22)R | CTTCCCTACCTTGGGCACC | 75 |
| LHβ | LH (−950)F | ATCTGAACTCTGCTGCACTT | 76 |
| LHβ | LH (−700)R | TGTACTGGGCAAATTCGGTC | 77 |
| LHβ | LH (−401)R | GCACCACCGTCTCCCAGCCC | 78 |
| LHβ | LH −284F | GACGTTTACTCCAGCAATCTGG | 79 |
| GnRHR | mGnRHR (−900)F | GTAATTGGTAATCTGATAACAAC | 80 |
| GnRHR | mGnRHR (−750)R | ATCCTATTGTGTTATATAACCAC | 81 |
| GnRHR | mGnRHR (−579)F | TTCAGCATGGATAGTCTGATC | 82 |
| GnRHR | mGnRHR (−468)R | ACGACGGACTTATAGCTAGTA | 83 |
| GnRHR | GnRHR −353F | GTATCTGTCTAGTCACAACAG | 84 |
| GnRHR | GnRHR −160R | GAGTGCTAACAGAACATGCC | 85 |
| GnRHR | GnRHR −255F | GTCCTTCCTCACCTACGATA | 86 |
| GnRHR | GnRHR −99R | GATACTCTGGACTTAACGTGA | 87 |

Similarly methylated DNA immunoprecipitation (MeDIP) was performed with antibody to 5mC DNA (Diagenode) 5 μg antibody for 5 μg sonicated genomic DNA.

The delivery delivery proteins—the minimal delivery protein (MDP) chimeric protein (protamine and NLS, SEQ ID NO: 89) was synthesized by GL Biochem. It was crosslinked to GnRH Lys6 analog (SEQ ID NO: 9, Sigma), via the Lys⁶ of the GnRH and the C-terminal Cys of the above protein using sulfo-GMBS. The crosslinked protein was dialyzed against PBS. NF51 ([stearyl-AGYLLG]-delta-OINLKALAALAKKIL, SEQ ID NO: 103 conjugated through Gly6 to the δ-NH2 group of ornithine 1 of SEQ ID NO: 100) was purchased commercially (Icosagen) and cross-linked to GnRH Lys6 using Traut reagent (Pierce) followed by sulfo-GMBS (Thermo Fisher). Peptide 1 comprises the same CPP sequence as NF51 with a terminal cysteine (stearyl-AGYLLG)-delta-OINLKALAALAKKILC-NH2, SEQ ID NO: 103 conjugated through Gly6 to the δ-NH2 group of ornithine 1 of SEQ ID NO: 101) for direct cross-linking, and Peptide 2 contains also the PACE cleavage (stearyl-AGYLLG)-delta-OINLKALAALAKKILRVRRC-NH2, SQ ID NO: 103 conjugated through Gly6 to the δ-NH2 group of ornithine 1 of SEQ ID NO: 102). The terminal cysteine was crosslinked directly to the GnRH Lys6 similarly using sulfo-GMBS. Each of these delivery proteins were mixed with the DNA at a ratio of 5:1 (5 μg peptide to 1 μg DNA) and incubated for 5 minutes at room temperature before addition to the cells in media that lacked serum and antibiotics.

EtBr displacement assay—EtBr displacement assay was performed using in a 50 μl reaction volume in HBS buffer, 100-1000 ng protein and 200 ng plasmid DNA. Following 10 minutes incubation at room temperature, 1 μl EtBr (8 ng/μl) was added for a further 10 minutes before OD measurements were read (320 nm ex/615 nm em). Values were calculated relative to substrate blank (no DNA) and displacement (no protein).

PACE cleavage assay—For the PACE cleavage assay, cells were washed twice with PBS, and media with no FCS was added for 3 hours. The incubation media was then collected and filtered (0.2 m), and concentrated (×5) using centricon filter (Millipore) and either assayed immediately or frozen at 50% glycerol in −80° C. For the assay, 100 μl media was added to 100 μl buffer (to a final reaction concentration: 100 mM HEPES PH=7.5, 0.5% Triton, 1 mM CaCl₂), 1 mM β-mercaptoethanol) together with 25 μM Boc-arg-val-arg-arg-MCA peptide (SEQ ID NO: 96, 3155-v by Peptide institute). Following 3 hours in the dark at 30° C., the reaction was stopped by adding ZnCl2 (1 mM final concentration), and OD measurements were taken (380 nm ex/460 nm em). Values were calculated relative to negative controls that lacked the peptide.

Transfection with delivery peptides—Mixed cell cultures, in which the gonadotropes comprise ˜10% of the cells, as in the pituitary gland, were transferred to media without FCS or antibiotics and pre-incubated for 1.5 hours prior to transfection. The DNA: peptide mix (ratio 1:5; for every 1 μg DNA [mCherry or KRAB-Sf1], 5 μg peptide was used) was incubated for 5 minutes and then added to the cells. Following 1.5 hours the media was removed and replaced with fresh complete media. Cells were collected 24-48 hours later for counting by FACS to determine specificity and efficacy of the delivery, or after 2 weeks for gene expression analysis by qPCR. Further optimization in the mixed cell cultures utilized GnRH-crosslinked Peptide2 mixed 1:10 with non-crosslinked Peptide 2 to attain maximal efficiency and targeting specificity.

### Example 1

### Generation of a Chimeric Protein for Reducing Expression of Gonadotropins Through Chromatin Compaction

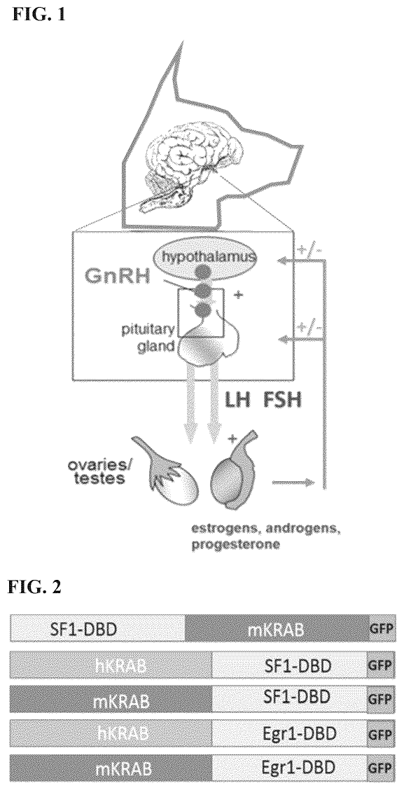

The gonadotropins, luteinizing hormone (LH) and follicle stimulating hormone (FSH), regulate reproductive development and function through binding to receptors in the gonads to stimulate their growth, gametogenesis and steroidogenesis. The levels of gonadotropin synthesis and secretion are highly dynamic, and this precise control is crucial in enabling reproductive function. This control is exerted through a complex network of hormones and feedback along the hypothalamic-pituitary-gonadal axis, culminating in diverse signals that act on, and interact in, pituitary gonadotropes (FIG. 1).

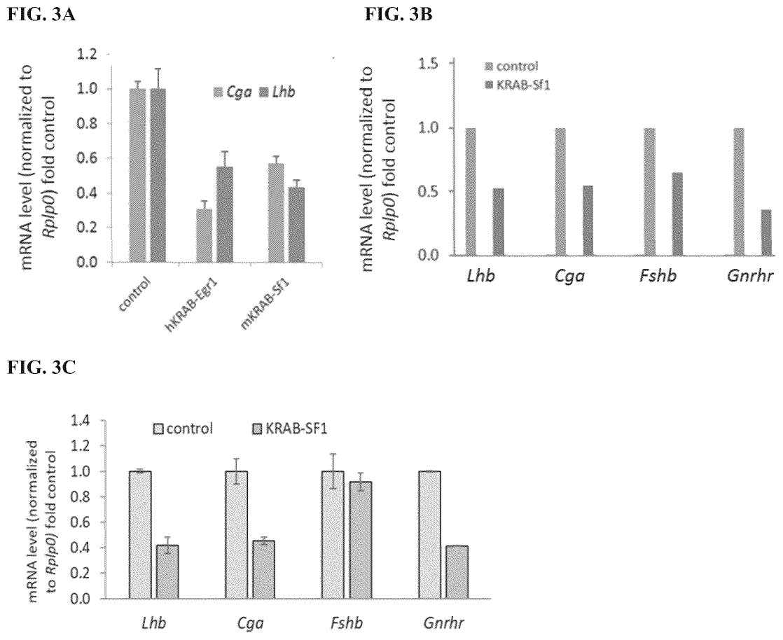

As a novel method for contraception, a number of chimeric DNA constructs containing sequences encoding the DNA binding domain (DBD) of various transcription factors (Egr-1, Sf-1), which recognize the gonadotropin genes Cga, Lhb and Fshb, fused N-terminal or C-terminal to the chromatin modifying KRAB domain [mouse KRAB (mKRAB), human KRAB (hKRAB)] were constructed (FIG. 2). The constructs also encoded GFP for identification of the transfected cells (FIG. 2). The constructs were transiently transfected into an immature αT3-1 gonadotrope cell line which expresses the gonadotropins Cga and Lhb. Two weeks following transfection (8 duplications) Cga and Lhb mRNA levels in GFP-positive cells were measured by quantitative real-time PCR (qPCR). The constructs containing the KRAB domain, fused to Sf-1 or Egr-1 DBD reduced Cga and Lhb mRNA levels (FIG. 3A). The efficacy of the constructs containing the KRAB domain fused to Sf-1 DBD in both orientations was also tested in a different LβT2 gonadotrope cell line which expresses Lhb more abundantly and also expresses low levels of Fshb. These studies, carried out 12 days following transfection, confirmed that both constructs reduced expression of the gonadotropin genes and also of the Gnrhr gene. Specifically, >50% repression of expression of the Lhb, Cga and Gnrhr genes was apparent, while Fshb appeared less sensitive (FIGS. 3B-C).

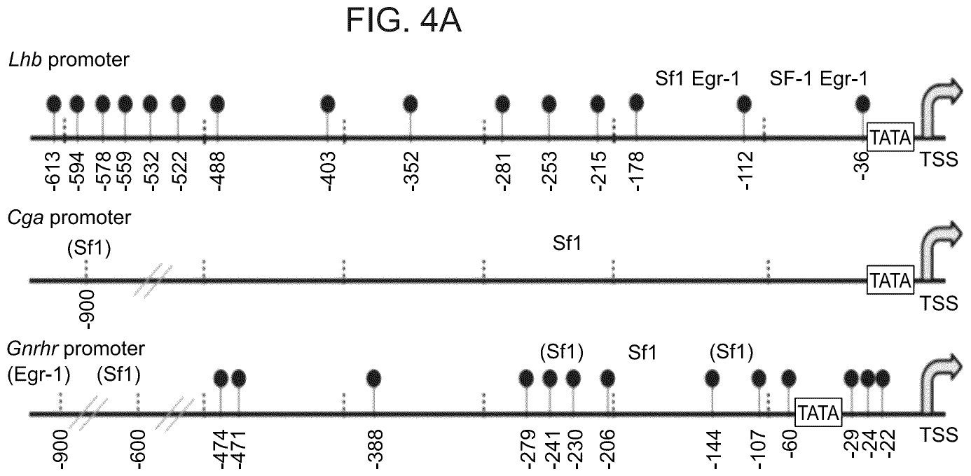

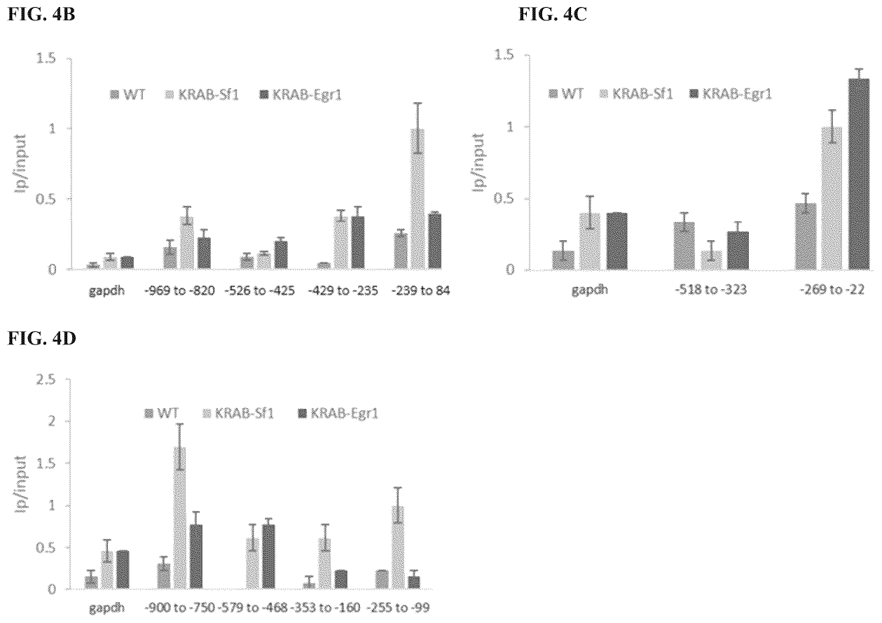

To verify that the demonstrated effects on expression reflect binding at the expected location of the relevant genes, a ChTP analysis for GFP was effected. The ChTP analysis assessed GFP localization on the Cga, Lhb and Gnrhr genes in αT3-1 gonadotrope cells transfected with the chimeric protein KRAB-Sf-1 or KRAB-Egr-1 as compared to that in wild type (WT) cells that were not transfected, as control (FIGS. 4A-D). Both chimeric proteins clearly bound the Cga gene proximal promoter, and the KRAB-Sf-1 also bound at an additional site further upstream, in accordance with the literature and the presence of putative binding motifs (FIGS. 4A-B). For the Lhb gene, even though the chromatin is closed at this gene promoter in these cells [5] a strong signal was evident at the TSS in cells expressing the chimeric proteins as compared to the WT non-transfected control cells, and compared to the levels at the negative control gene, gapdh (FIG. 4C). Both factors are known to bind this region of the Lhb proximal promoter (FIG. 4A) and this finding confirms that the chimeric proteins can bind heterochromatin DNA. Also for Gnrhr there were a number of regions with strong signals (FIG. 4D), which correlate with the location of the reported binding sites, or sequence motifs at −920 (Egr-1), -611, -240-196 and -129 (Sf-1) (FIG. 4A); notably these proteins interact with each other, so binding by both proteins (or parts thereof, as in the chimeric proteins) at any of these sites is likely (due to the endogenous proteins present).

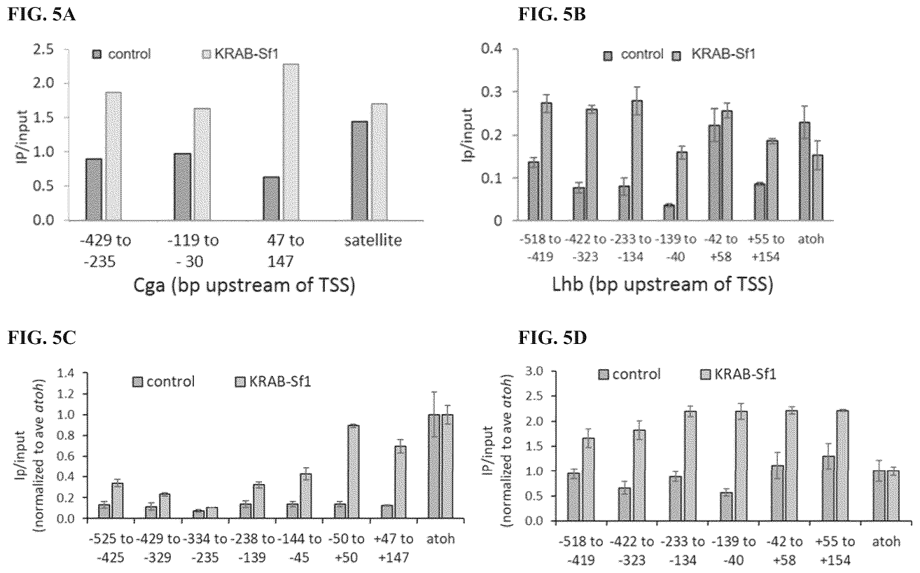

In the next step, the effect of the generated KRAB-Sf-1 construct on the chromatin, which would mediate lasting and progressive effects to repress gene expression, was tested. Initially αT3-1 cells were stably transfected in order to verify whether these constructs do in fact induce chromatin compaction. In these cells, the KRAB-Sf-1 construct increased compaction of the Cga promoter DNA, as demonstrated by higher levels of histone H3 determined by chromatin immunoprecipitation for histone H3 (FIG. 5A-C). A similar though slightly less potent effect was apparent on the Lhb gene proximal promoter, in both αT3-1 and LβT2 cells, which is not surprising given that this gene is normally packaged more tightly than the Cga gene [5] (FIGS. 5B and 5D). In addition, 5 weeks following transient transfection of the KRAB-Sf-1 construct, increased H3 association with both gene promoters was evident, while no increase was detected at the Atoh control gene which is not an Sf-1 target (FIG. 5E). Taken together, the KRAB-Sf-1 construct is clearly capable of altering the chromatin packaging specifically at the Cga and Lhb genes.

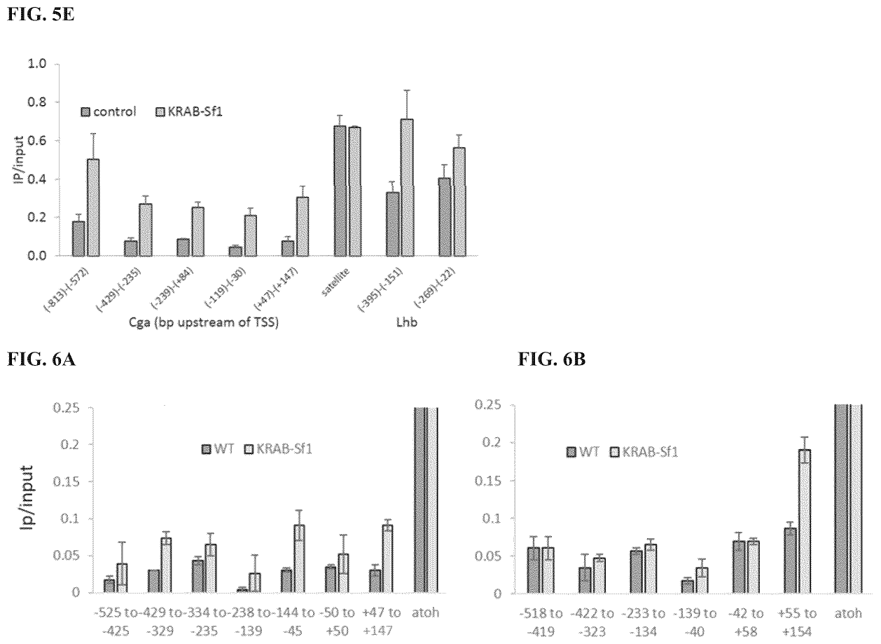

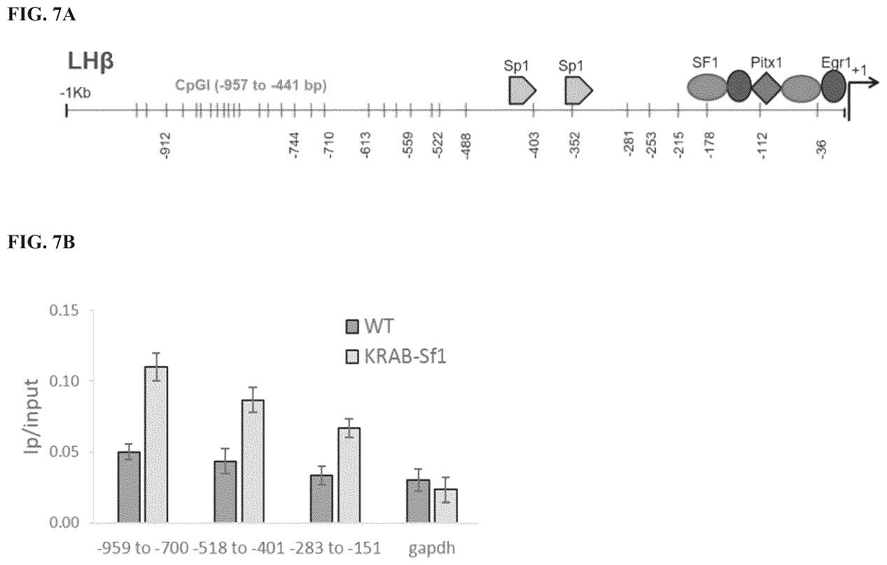

Subsequently, ChIP analysis for a number of repressive histone modifications that were anticipated to be induced by binding of the KRAB domain at a particular locus was performed. KRAB-Sf-1 elevated the levels of H3K9me3 on the Cga proximal promoter, but appeared to have little effect on the Lhb promoter although H3K9me3 was elevated markedly at the start of the gene itself (FIG. 6A-B). The Lhb gene promoter, contrary to Cga, is regulated by DNA methylation, and CpG methlyation levels are higher in αT3-1 compared to LβT2 cells, correlating with its expession levels [6]. Therefore, changes in its DNA methylation levels in LβT2 cells were assessed following expression of the KRAB-Sf-1 construct. MeDTP analysis was carried out at the promoter and further upstream, covering CpG-rich regions that can be methylated (FIG. 7A). The KRAB-Sf-1 construct clearly elevated DNA methlyation in these regions (FIG. 7B), in accordance with the numbers of CpGs present, and reached more than 2-fold the level of that in the control cells, which is similar to the levels seen previously in αT3-1 cells [6].

### Example 2

### Generation of a GnRH Based Targeted Delivery of a Nucleic Acid Construct

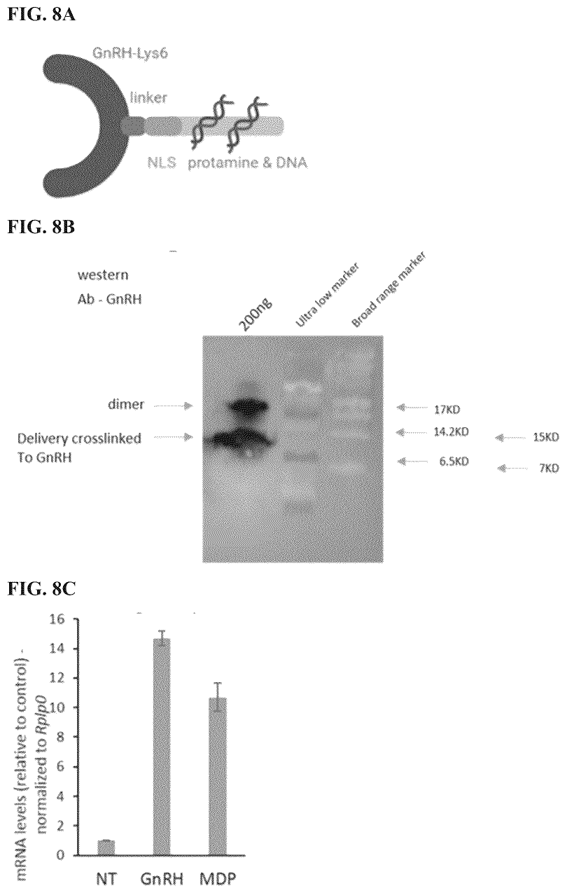

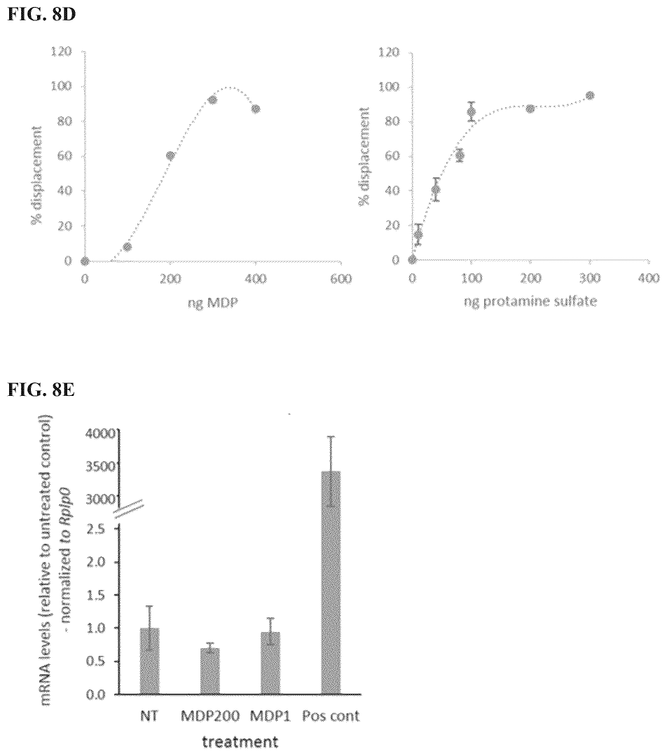

Gonadotrophin releasing hormone (GnRH) receptors are G-protein-coupled receptors expressed primarily in the pituitary gonadotropes. Hence, the present inventors devised the use of GnRH for targeted delivery of the constructs described in Example 1 hereinabove. To this end, a number of designs for a delivery protein, in which GnRH was cross-linked to a peptide that could package DNA and transport it into the cell (e.g. FIG. 8A) were tested. A minimal delivery protein (MDP) comprising a GnRH analog [(GnRH-Lys6: EHWSYKLRPG (SEQ ID NO: 9)] cross-linked (FIG. 8B) to the cell penetrating peptide (CPP) protamine, together with a nuclear localization signal (NLS) was successfully generated. This delivery protein could bind the GnRH receptor (GnRHR), as seen by activation of Egr-1 (FIG. 8C). It also packaged the DNA construct efficiently, as seen in an EtBr displacement assay (FIG. 8D). However the construct was not transferred into the cells (FIG. 8E).

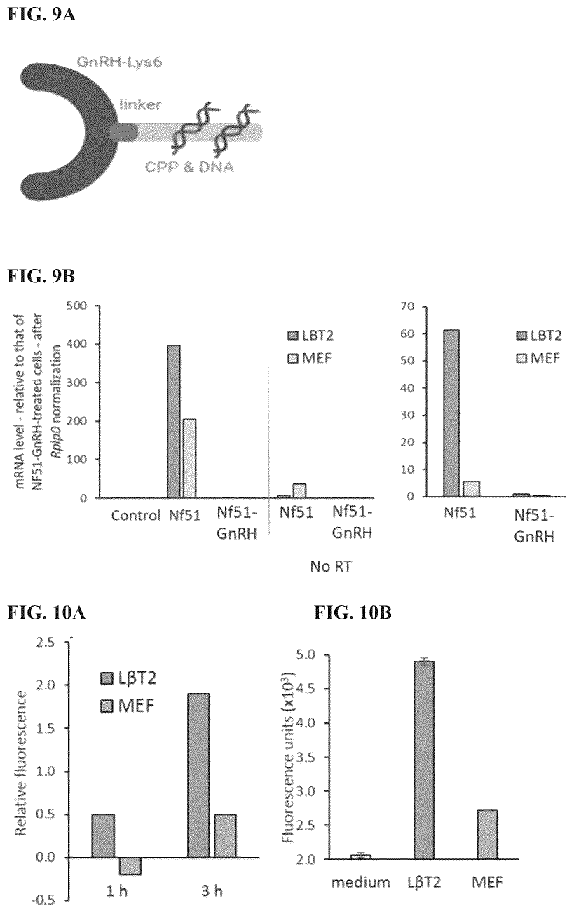

In order to examine the properties of a different CPP, NF51, it was packaged with the DNA construct and was applied to LβT2 gonadotrope or MEF cells, with or without cross-linking to GnRH (FIG. 9A) which was performed in 2-steps with Traut reagent and then sulfo-GMBS. In the absence of cross-linking to GnRH, the CPP was highly efficient at transfecting both cell types, however the cross-linking to GnRH abolished this ability (FIG. 9B). This suggested that cross-linking of the CPP using this approach hinders its entry to the cell, so that dissociation of the CPP from the GnRH might facilitate delivery of the DNA construct.

Taking these observations into account, the present inventors took advantage of an endogenous protease, PACE4 that is secreted at very high levels from pituitary cells [7]. Fluorimetric assays using a substrate (Boc-Arg-Val-Arg-Arg-MCA, SEQ ID NO: 96) that contains the PACE4 cleavage motif, and media in which gonadotrope cells had been incubated were performed. In this assay, cleavage is detected by release of the fluorophore, measured at 380 nm excitation, 460 nm emission. The gonadotrope cells secreted significant amounts of the enzyme, cutting the reporter and emitting fluorescence at a much higher level than seen in the incubation media of MEF cells (FIG. 10A-B).

In view of the above, embodiments of the invention suggest a novel delivery vehicle comprising a GnRH receptor binding protein cross linked to a CPP attached to a polynucleotide encoding a therapeutic agent via a linker comprising a cleavage motif that can be cleaved by a proteolytic enzyme secreted from the target cell. This peptide (Peptide 2, SEQ ID NO: 102) contains a terminal cysteine for specific and direct cross-linking to Lys6 of the GnRH analog via GMBS. FIGS. 11A-E show schematic representations of such a delivery vehicle using as an example a GnRH-Lys6 analog attached via its Lys6, to a CPP that packages the DNA encoding the fusion protein described in Example 1 hereinabove for targeted delivery to pituitary cells. The short linker between the GnRH and the CPP contains a cleavage motif that is optimally cleaved by a proteolytic enzyme, PACE4, which is secreted by the pituitary cells (FIGS. 10A-B; [7]). In this way, GnRH delivers the CPP+DNA to the gonadotrope cell surface, where GnRH binds its receptor. At the cell surface, the proteolytic enzyme secreted from the cell will cleave the CPP from the GnRH, which allows the CPP to transport the DNA into the cells (FIG. 11C). Inside the cells, the chimeric protein, e.g. KRAB-Sf-1 encoded by the construct (FIG. 11B), is expressed and binds Sf-1 regulated genes, to which it recruits the repressive epigenetic machinery to close the chromatin (FIGS. 11D-E). Subsequently, the cells' endogenous epigenetic machinery recognizes this closed chromatin state and maintains the locus repressed, even after the original signal is no longer present (FIG. 11E). This inhibition, by reducing gonadotropin gene expression, should lower also the circulating levels of the encoded hormones that are required for reproductive function.

### Example 3

### Specific Targeting of a Polynucleotide to GnRH Receptor Expressing Cells

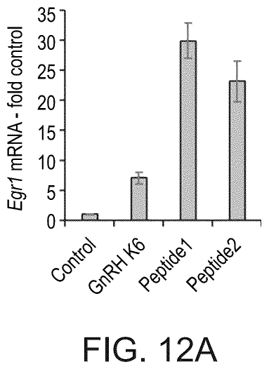

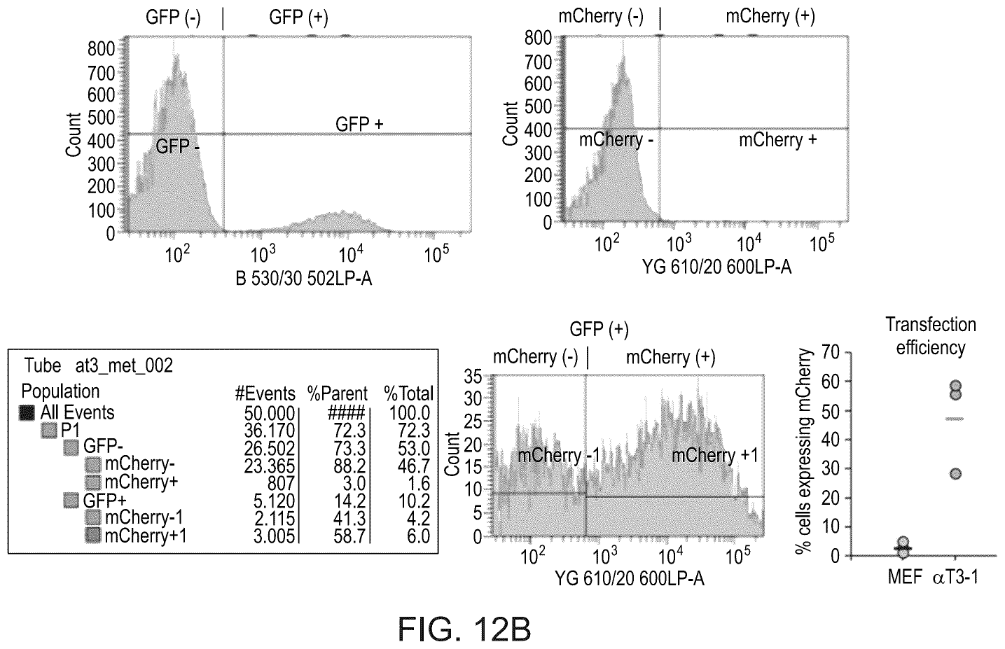

To test whether the newly designed GnRH-carrier might deliver DNA specifically to GnRH receptor-expressing cells, it was first applied to αT3-1 gonadotrope cells and Egr1 mRNA levels measured as a readout for GnRHR binding. GnRH Lys6 cross-linked to this peptide (Peptide 2, SEQ ID NO: 102) activated Egr1 mRNA, indicating that this form of cross-linking maintains GnRHR binding. GnRH crosslinked to same peptide without the PACE cleavage site (Peptide 1, SEQ ID NO: 101) also activated the receptor (FIG. 12A). Both peptides could deliver some DNA into the cells, but Peptide 2 which includes the PACE4 cleavage site was much more specific to gonadotropes. GnRH Lys6 was thus crosslinked to Peptide 2, loaded with mCherry-encoding DNA, and applied to αT3-1 gonadotrope cells that stably express GFP, mixed with MEFs (which do not express the GnRH receptor). Following, cells were sorted and counted by FACS to determine the number of cells expressing the delivered DNA in an unbiased manner. After optimization, the number of gonadotrope cells that were transfected reached around 58%, while only 3% of MEF cells expressed the mCherry (FIG. 12B). Assuming 80% of the αT3-1 indeed express the GnRH receptor, the targeting was successful in 72.5% of the GnRH receptor-expressing cells.

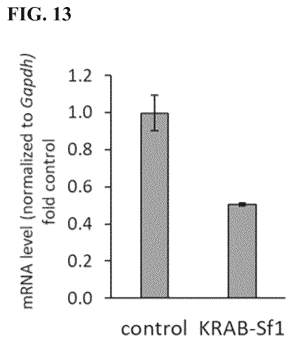

Following, the GnRH-based delivery vehicle loaded with a DNA encoding KRAB-Sf1 or GFP (control) was applied for 1.5 hours to mixed cell cultures comprising murine αT3-1 gonadotrope cells and human non-pituitary cells (˜1:10). After 13 days (˜7 cell-duplications), cells were harvested, RNA extracted and qPCR performed. A ˜50% drop in Cga mRNA levels was seen relative to levels in control cells at this time (FIG. 13).

### Example 4

### Cancerous Pancreatic Cells Secrete Functional PACE4

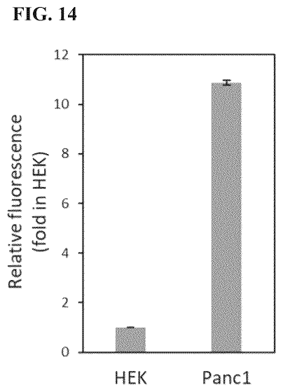

Numerous scientific papers report that PACE4 and similar proteolytic enzymes are produced in large amounts specifically in cancer cells [e.g. Bassi D E, et al., Mol Carcinog. 2000 June; 28(2):63-9; Mercapide J, et al. Clin Cancer Res. 2002; 8(6):1740-1746; Bassi D E et al. Mol Carcinog. 2001; 31(4):224-232; Page R E, et al. Cell Oncol. 2007]. In order to verify that pancreatic cancer cells indeed secrete sufficient PACE4 to cleave the delivery peptide, fluorometric assays using the Boc-Arg-Val-Arg-Arg-MCA (SEQ ID NO: 96) substrate that contains this cleave domain and emits a fluorescent signal upon cleavage were effected. To this end, the PANC-1 pancreatic cell line or HEK (human embryonic kidney) cells were cultured, rinsed and the medium was collected after 3 hours before incubation with the reporter peptide, Boc-Arg-Val-Arg-Arg-MCA (SEQ ID NO: 96). Measurement of the fluorescence confirmed that the PANC1 pancreatic cells secrete significant amounts of the enzyme, cutting the reporter and emitting fluorescence at a much higher levels than the HEK cells (FIG. 14).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Waldmann T, Schneider R. (2013) Targeting histone modifications-epigenetics in cancer. Curr Opin Cell Biol. 25(2):184-9.
2. Zhao L, Duan Y T, Lu P, Zhang Z J, Zheng X K, Wang J L, Feng W S. (2018) Epigenetic Targets and their Inhibitors in Cancer Therapy. Curr Top Med Chem. 18(28):2395-2419.
3. Melamed P. (2008) Histone deacetylases and repression of the gonadotropin genes. Trends Endocrinol Metab. 19(1):25-31
4. Zhao L, Bakke M, Krimkevich Y, Cushman L J, Parlow A F, Camper S A, Parker K L. (2001) Steroidogenic factor 1 (SF1) is essential for pituitary gonadotrope function. Development. 128(2):147-54
5. Rudnizky S, Bavly A, Malik O, Pnueli L, Melamed P, Kaplan A. (2016) DNA sequence and H2A. Z control the stability and mobility of promoter nucleosomes to regulate basal expression of the LH genes. Nature Communications 7:12958.
6. Yosefzon Y, Tsukerman A, Pnueli L, Sen Q, Boehm U, Melamed P. (2017) Repression of a novel truncated Tet1 isoform is required for the central activation of reproduction. Proc. Natl. Acad. Sci. USA 114:10131-6
7. Mains R E, Berard C A, Denault J B, Zhou A, Johnson R C, Leduc R. (1997) PACE4: a subtilisin-like endoprotease with unique properties. Biochem J. 321:587-93

1. A composition comprising a polypeptide comprising a GnRH receptor binding protein and a polynucleotide coding a therapeutic agent, wherein said polypeptide is in association with said polynucleotide via a linker comprising a cleavage motif that can be cleaved by a proteolytic enzyme secreted by a cell expressing said GnRH receptor.

2. The composition of claim 1, wherein said polynucleotide is attached to- or encapsulated by a cell penetrating moiety.

3. A method of treating a medical condition in a subject in need thereof, the method comprising administering to the subject a therapeutically effective amount of the composition of claim 1, wherein cells associated with said medical condition express said GnRH receptor and secrete said proteolytic enzyme, thereby treating the medical condition in the subject.

4-5. (canceled)

6. The method claim 3, wherein said medical condition is cancer.

7-8. (canceled)

9. A nucleic acid construct comprising a nucleic acid sequence encoding a fusion protein comprising a DNA binding domain (DBD) of a transcription factor that binds a regulatory region of a gonadotropin gene attached to a chromatin modifying domain that represses transcription upon association with said DBD.

10. The nucleic acid construct of claim 9, wherein said transcription factor is selected from the group consisting of Sf-1, Egr-1 and Nur77.

11-13. (canceled)

14. The nucleic acid construct of claim 10, wherein said chromatin modifying domain is a KRAB domain, catalytic domain of DNMT3a or HP1α.

15-16. (canceled)

17. A composition comprising the nucleic acid construct of claim 9, attached to- or encapsulated by a cell penetrating moiety.

18. (canceled)

19. A composition comprising the nucleic acid construct of claim 9 attached to a gonadotrope-targeting moiety.

20. The composition of claim 17, comprising a gonadotrope-targeting moiety attached to said nucleic acid construct or said cell penetrating moiety.

21. The composition of claim 20, wherein said gonadotrope-targeting moiety is a GnRH receptor binding protein.

22. The composition of claim 20, wherein said gonadotrope-targeting moiety is attached to said nucleic acid construct or said composition using a linker.

23. The composition of claim 22, wherein said linker comprises a cleavage motif that can be cleaved by a proteolytic enzyme secreted from the pituitary.

24. The composition of claim 1, wherein said proteolytic enzyme is selected from the group consisting of PACE4, prohormone convertases (PC)1/3, PC2, PC4 PC5/6 and furin.

25. The composition of claim 1, wherein said proteolytic enzyme is selected from the group consisting of MMP, ADAM, ADAMTS, Kallikreins, PSA, uPA, Furin, PCSK, trypsin-like protease and thrombin.

26. (canceled)

27. A composition comprising a GnRH receptor binding protein attached to a cell penetrating peptide by a linker comprising a cleavage motif that can be cleaved by PACE4, wherein said cell penetrating peptide is attached to- or encapsulates a nucleic acid construct encoding a fusion protein comprising a DNA binding domain (DBD) of Sf-1 attached to a KRAB domain.

28-29. (canceled)

30. The composition of claim 1, wherein said therapeutic agent is a contraceptive.

31. (canceled)

32. A method of contraception in a mammal, the method comprising administering to the mammal an effective amount of the nucleic acid construct claim 9, thereby effecting contraception in the mammal.

33-34. (canceled)

35. A method of decreasing or preventing sex-steroid driven behavior in a non-human mammal, the method comprising administering to the non-human mammal an effective amount of the nucleic acid construct claim 9, thereby decreasing or preventing the sex-steroid driven behavior in the non-human mammal.

36-40. (canceled)

41. A method of treating a medical condition selected from the group consisting of steroid-dependent cancer, uterine infection, uterine fibroids and endometriosis in a subject in need thereof, the method comprising administering to the subject a therapeutically effective amount of the nucleic acid construct of claim 9, thereby treating the medical condition in the subject.

42-48. (canceled)


SEQUENCE LISTING (103 sequences)

SEQ ID NO: 1 - protein, 111 aa, Mus musculus
MDYSYDEDLDELCPVCGDKVSGYHYGLLTCESCKGFFKRTVQNNKHYTCTESQSCKIDKTQRKRCPFCRFQKCLTVGMRLEAVRADRMRGGRNKFGPMYKRDRALKQQKKA

SEQ ID NO: 2 - DNA, 333 bp, Mus musculus
  CDS 1..333
  source 1..333: mol_type = other DNA
atggactattcgtacgacgaggacctggacgagctgtgtccagtgtgtggtgacaaggtgtcgggctaccactacgggctgctcacgtgcgagagctgcaagggcttcttcaagcgcacagtccagaacaacaagcattacacgtgcaccgagagtcagagctgcaaaatcgacaagacgcagcgtaagcgctgtcccttctgccgcttccagaagtgcctgacggtgggcatgcgcctggaagctgtgcgtgctgatcgaatgcggggtggccggaacaagtttgggcccatgtacaagagagaccgggccttgaagcagcagaagaaagca

SEQ ID NO: 3 - protein, 86 aa, Mus musculus
YACPVESCDRRFSRSDELTRHIRIHTGQKPFQCRICMRNFSRSDHLTTHIRTHTGEKPFACDICGRKFARSDERKRHTKIHLRQKD

SEQ ID NO: 4 - DNA, 258 bp, Mus musculus
  misc_feature 1..258: note = mouse Egr1 DBD nucleic acid sequence
  source 1..258: mol_type = other DNA
tatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccaggagtgatgaacgcaagaggcataccaaaatccatttaagacagaaggac

SEQ ID NO: 5 - protein, 89 aa, Mus musculus
MAPRPPTAKPQESVTFKDVAVNFTQEEWHHVGPAQRSLYRDVMLENYNHLVSLGYQVSKPEVIFKLEQGEEPWISEKEIQRPFCPDWKT

SEQ ID NO: 6 - protein, 90 aa, Homo sapiens
MDAKSLTAWSRTLVTFKDVFVDFTREEWKLLDTAQQIVYRNVMLENYKNLVSLGYQLTKPDVILRLEKGEEPWLVEREIHQETHPDSETA

SEQ ID NO: 7 - DNA, 267 bp, Mus musculus
  misc_feature 1..267: note = mouse KRAB domain nucleic acid sequence
  source 1..267: mol_type = other DNA
atggccccgagacctccgacggccaagccccaggagtcggtgacattcaaagatgtggctgtgaacttcacccaggaagaatggcaccacgtgggccctgcccagaggagcttatacagggatgtcatgctggagaactacaaccacctggtgtcgctcgggtatcaagtctccaagccagaggtgatcttcaaattggagcaaggagaagagccatggatatcagagaaagaaatccaaagacctttctgtccagactggaagacc

SEQ ID NO: 8 - DNA, 270 bp, Homo sapiens
  misc_feature 1..270: note = human KRAB domain nucleic acid sequence
  source 1..270: mol_type = other DNA
atggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgca

SEQ ID NO: 9 - protein, 10 aa, synthetic construct
  REGION 1..10: note = GnRH analog amino acid sequence
EHWSYKLRPG

SEQ ID NO: 10 - protein, 96 aa, Homo sapiens
MCLRMKPIQKLLAGLILLTWCVEGCSSQHWSYGLRPGGKRDAENLIDSFQEIVKEVGQLAETQRFECTTHQPRSPLRDLKGALESLIEEETGQKKI

SEQ ID NO: 11 - protein, 92 aa, Homo sapiens
MKPIQKLLAGLILLTWCVEGCSSQHWSYGLRPGGKRDAENLIDSFQEIVKEVGQLAETQRFECTTHQPRSPLRDLKGALESLIEEETGQKKI

SEQ ID NO: 12 - DNA, 26 bp, synthetic construct
  misc_feature 1..26: note = Single strand DNA oligonucleotide
  source 1..26: mol_type = other DNA
aagcttatggactattcgtacgacga

SEQ ID NO: 13 - DNA, 28 bp, synthetic construct
  misc_feature 1..28: note = Single strand DNA oligonucleotide
  source 1..28: mol_type = other DNA
ggtacccctgctttcttctgctgcttca

SEQ ID NO: 14 - DNA, 28 bp, synthetic construct
  misc_feature 1..28: note = Single strand DNA oligonucleotide
  source 1..28: mol_type = other DNA
aagcttatgtatgcttgccctgtcgagt

SEQ ID NO: 15 - DNA, 29 bp, synthetic construct
  misc_feature 1..29: note = Single strand DNA oligonucleotide
  source 1..29: mol_type = other DNA
ggtaccccgtccttctgtcttaaatggat

SEQ ID NO: 16 - DNA, 24 bp, synthetic construct
  misc_feature 1..24: note = Single strand DNA oligonucleotide
  source 1..24: mol_type = other DNA
ctcgagatggccccgagacctccg

SEQ ID NO: 17 - DNA, 26 bp, synthetic construct
  misc_feature 1..26: note = Single strand DNA oligonucleotide
  source 1..26: mol_type = other DNA
aagcttggtcttccagtctggacaga

SEQ ID NO: 18 - DNA, 26 bp, synthetic construct
  misc_feature 1..26: note = Single strand DNA oligonucleotide
  source 1..26: mol_type = other DNA
ctcgagatggatgctaagtcactaac

SEQ ID NO: 19 - DNA, 26 bp, synthetic construct
  misc_feature 1..26: note = Single strand DNA oligonucleotide
  source 1..26: mol_type = other DNA
aagctttgcagtctctgaatcaggat

SEQ ID NO: 20 - DNA, 25 bp, synthetic construct
  misc_feature 1..25: note = Single strand DNA oligonucleotide
  source 1..25: mol_type = other DNA
atggattactacagaaaatatgcag

SEQ ID NO: 21 - DNA, 25 bp, synthetic construct
  misc_feature 1..25: note = Single strand DNA oligonucleotide
  source 1..25: mol_type = other DNA
cctgaataataaagtctccatcagg

SEQ ID NO: 22 - DNA, 20 bp, synthetic construct
  misc_feature 1..20: note = Single strand DNA oligonucleotide
  source 1..20: mol_type = other DNA
ctagcatggtccgagtactg

SEQ ID NO: 23 - DNA, 20 bp, synthetic construct
  misc_feature 1..20: note = Single strand DNA oligonucleotide
  source 1..20: mol_type = other DNA
ctgagggctacaggaaagga

SEQ ID NO: 24 - DNA, 22 bp, synthetic construct
  misc_feature 1..22: note = Single strand DNA oligonucleotide
  source 1..22: mol_type = other DNA
gctggagagcaatctgctgcca

SEQ ID NO: 25 - DNA, 23 bp, synthetic construct
  misc_feature 1..23: note = Single strand DNA oligonucleotide
  source 1..23: mol_type = other DNA
tattgggccgagctgggtcctta

SEQ ID NO: 26 - DNA, 20 bp, synthetic construct
  misc_feature 1..20: note = Single strand DNA oligonucleotide
  source 1..20: mol_type = other DNA
ccacagtcttctcgcaatgt

SEQ ID NO: 27 - DNA, 20 bp, synthetic construct
  misc_feature 1..20: note = Single strand DNA oligonucleotide
  source 1..20: mol_type = other DNA
accaaatgcctaggacatag

SEQ ID NO: 28 - DNA, 20 bp, synthetic construct
  misc_feature 1..20: note = Single strand DNA oligonucleotide
  source 1..20: mol_type = other DNA
gcgacctggaagtccaacta

SEQ ID NO: 29 - DNA, 19 bp, synthetic construct
  misc_feature 1..19: note = Single strand DNA oligonucleotide
  source 1..19: mol_type = other DNA
atctgcttggagcccacat

SEQ ID NO: 30 - DNA, 20 bp, synthetic construct
  misc_feature 1..20: note = Single strand DNA oligonucleotide
  source 1..20: mol_type = other DNA
aacaaccctacgagcacctg

SEQ ID NO: 31 - DNA, 20 bp, synthetic construct
  misc_feature 1..20: note = Single strand DNA oligonucleotide
  source 1..20: mol_type = other DNA
agcggccagtataggtgatg

SEQ ID NO: 32 - DNA, 20 bp, synthetic construct
  misc_feature 1..20: note = Single strand DNA oligonucleotide
  source 1..20: mol_type = other DNA
ggaagcagcattcaggtctc

SEQ ID NO: 33 - DNA, 24 bp, synthetic construct
  misc_feature 1..24: note = Single strand DNA oligonucleotide
  source 1..24: mol_type = other DNA
caggataggactcagggaatacag

SEQ ID NO: 34 - DNA, 19 bp, synthetic construct
  misc_feature 1..19: note = Single strand DNA oligonucleotide
  source 1..19: mol_type = other DNA
ccctcactcaggtcgcctg

SEQ ID NO: 35 - DNA, 18 bp, synthetic construct
  misc_feature 1..18: note = Single strand DNA oligonucleotide
  source 1..18: mol_type = other DNA
cgtgcgaggagccaatca

SEQ ID NO: 36 - DNA, 24 bp, synthetic construct
  misc_feature 1..24: note = Single strand DNA oligonucleotide
  source 1..24: mol_type = other DNA
gacgacttgaaaaatgacgaaatc

SEQ ID NO: 37 - DNA, 24 bp, synthetic construct
  misc_feature 1..24: note = Single strand DNA oligonucleotide
  source 1..24: mol_type = other DNA
catattccaggtccttcagtgtgc

SEQ ID NO: 38 - DNA, 21 bp, synthetic construct
  misc_feature 1..21: note = Single strand DNA oligonucleotide
  source 1..21: mol_type = other DNA
tatatgtacacatctgtgggt

SEQ ID NO: 39 - DNA, 21 bp, synthetic construct
  misc_feature 1..21: note = Single strand DNA oligonucleotide
  source 1..21: mol_type = other DNA
ggttaagagcatgaactgctc

SEQ ID NO: 40 - DNA, 22 bp, synthetic construct
  misc_feature 1..22: note = Single strand DNA oligonucleotide
  source 1..22: mol_type = other DNA
cacacctggacatatctactgt

SEQ ID NO: 41 - DNA, 26 bp, synthetic construct
  misc_feature 1..26: note = Single strand DNA oligonucleotide
  source 1..26: mol_type = other DNA
caattgctccaatttcttaaattaac

SEQ ID NO: 42 - DNA, 24 bp, synthetic construct
  misc_feature 1..24: note = Single strand DNA oligonucleotide
  source 1..24: mol_type = other DNA
gccaaatgctctctcttcataagc

SEQ ID NO: 43 - DNA, 22 bp, synthetic construct
  misc_feature 1..22: note = Single strand DNA oligonucleotide
  source 1..22: mol_type = other DNA
cctctggactttttgagggatg

SEQ ID NO: 44 - DNA, 20 bp, synthetic construct
  misc_feature 1..20: note = Single strand DNA oligonucleotide
  source 1..20: mol_type = other DNA
ccaagtgccatccaatcact

SEQ ID NO: 45 - DNA, 22 bp, synthetic construct
  misc_feature 1..22: note = Single strand DNA oligonucleotide
  source 1..22: mol_type = other DNA
ccagcagagtaatacaaattgc

SEQ ID NO: 46 - DNA, 24 bp, synthetic construct
  misc_feature 1..24: note = Single strand DNA oligonucleotide
  source 1..24: mol_type = other DNA
gtggtcacaaatattttactcttt

SEQ ID NO: 47 - DNA, 18 bp, synthetic construct
  misc_feature 1..18: note = Single strand DNA oligonucleotide
  source 1..18: mol_type = other DNA
atcacctgcccagaacac

SEQ ID NO: 48 - DNA, 20 bp, synthetic construct
  misc_feature 1..20: note = Single strand DNA oligonucleotide
  source 1..20: mol_type = other DNA
agccatctctctagacccta

SEQ ID NO: 49 - DNA, 22 bp, synthetic construct
  misc_feature 1..22: note = Single strand DNA oligonucleotide
  source 1..22: mol_type = other DNA
gcagcctaagaattacttaagc

SEQ ID NO: 50 - DNA, 24 bp, synthetic construct
  misc_feature 1..24: note = Single strand DNA oligonucleotide
  source 1..24: mol_type = other DNA
ggagcaattgttttatttttctgt

SEQ ID NO: 51 - DNA, 25 bp, synthetic construct
  misc_feature 1..25: note = Single strand DNA oligonucleotide
  source 1..25: mol_type = other DNA
attagctaagtacctgatattttca

SEQ ID NO: 52 - DNA, 24 bp, synthetic construct
  misc_feature 1..24: note = Single strand DNA oligonucleotide
  source 1..24: mol_type = other DNA
gctaattaaatgtgctactcttag

SEQ ID NO: 53 - DNA, 25 bp, synthetic construct
  misc_feature 1..25: note = Single strand DNA oligonucleotide
  source 1..25: mol_type = other DNA
ggccattttctatgtaatattattg

SEQ ID NO: 54 - DNA, 20 bp, synthetic construct
  misc_feature 1..20: note = Single strand DNA oligonucleotide
  source 1..20: mol_type = other DNA
ggccaaatgctctctcttca

SEQ ID NO: 55 - DNA, 23 bp, synthetic construct
  misc_feature 1..23: note = Single strand DNA oligonucleotide
  source 1..23: mol_type = other DNA
ttgatcatatcacattgcaaccc

SEQ ID NO: 56 - DNA, 23 bp, synthetic construct
  misc_feature 1..23: note = Single strand DNA oligonucleotide
  source 1..23: mol_type = other DNA
gatcaattgatgtcatggtaatt

SEQ ID NO: 57 - DNA, 19 bp, synthetic construct
  misc_feature 1..19: note = Single strand DNA oligonucleotide
  source 1..19: mol_type = other DNA
aattgcatgttcccagctg

SEQ ID NO: 58 - DNA, 23 bp, synthetic construct
  misc_feature 1..23: note = Single strand DNA oligonucleotide
  source 1..23: mol_type = other DNA
gcaatttgtattactctgctggt

SEQ ID NO: 59 - DNA, 20 bp, synthetic construct
  misc_feature 1..20: note = Single strand DNA oligonucleotide
  source 1..20: mol_type = other DNA
gattgtcgatctgaggggaa

SEQ ID NO: 60 - DNA, 22 bp, synthetic construct
  misc_feature 1..22: note = Single strand DNA oligonucleotide
  source 1..22: mol_type = other DNA
gacacaccccttacttccagag

SEQ ID NO: 61 - DNA, 21 bp, synthetic construct
  misc_feature 1..21: note = Single strand DNA oligonucleotide
  source 1..21: mol_type = other DNA
ggatgcctggattgggtccaa

SEQ ID NO: 62 - DNA, 21 bp, synthetic construct
  misc_feature 1..21: note = Single strand DNA oligonucleotide
  source 1..21: mol_type = other DNA
atcctgattaggggctgggag

SEQ ID NO: 63 - DNA, 22 bp, synthetic construct
  misc_feature 1..22: note = Single strand DNA oligonucleotide
  source 1..22: mol_type = other DNA
ggcacagcttcaaccttaggtt

SEQ ID NO: 64 - DNA, 23 bp, synthetic construct
  misc_feature 1..23: note = Single strand DNA oligonucleotide
  source 1..23: mol_type = other DNA
ctcccctttaccttgtttcccgt

SEQ ID NO: 65 - DNA, 20 bp, synthetic construct
  misc_feature 1..20: note = Single strand DNA oligonucleotide
  source 1..20: mol_type = other DNA
cagggactagctccagtgtc

SEQ ID NO: 66 - DNA, 21 bp, synthetic construct
  misc_feature 1..21: note = Single strand DNA oligonucleotide
  source 1..21: mol_type = other DNA
tccctggcttccctgaccttg

SEQ ID NO: 67 - DNA, 20 bp, synthetic construct
  misc_feature 1..20: note = Single strand DNA oligonucleotide
  source 1..20: mol_type = other DNA
tgggggtggcaaggccacta

SEQ ID NO: 68 - DNA, 19 bp, synthetic construct
  misc_feature 1..19: note = Single strand DNA oligonucleotide
  source 1..19: mol_type = other DNA
ccacaacccgcaggtataa

SEQ ID NO: 69 - DNA, 19 bp, synthetic construct
  misc_feature 1..19: note = Single strand DNA oligonucleotide
  source 1..19: mol_type = other DNA
gattgggaagtaccctggg

SEQ ID NO: 70 - DNA, 22 bp, synthetic construct
  misc_feature 1..22: note = Single strand DNA oligonucleotide
  source 1..22: mol_type = other DNA
aatcccaccaaacccatagatg

SEQ ID NO: 71 - DNA, 24 bp, synthetic construct
  misc_feature 1..24: note = Single strand DNA oligonucleotide
  source 1..24: mol_type = other DNA
gtttatctcctgtttgaagtccac

SEQ ID NO: 72 - DNA, 22 bp, synthetic construct
  misc_feature 1..22: note = Single strand DNA oligonucleotide
  source 1..22: mol_type = other DNA
gcaccacctctggttggattga

SEQ ID NO: 73 - DNA, 22 bp, synthetic construct
  misc_feature 1..22: note = Single strand DNA oligonucleotide
  source 1..22: mol_type = other DNA
gtgtcccagtgaattggcctca

SEQ ID NO: 74 - DNA, 20 bp, synthetic construct
  misc_feature 1..20: note = Single strand DNA oligonucleotide
  source 1..20: mol_type = other DNA
caatctgggggttcagcgag

SEQ ID NO: 75 - DNA, 19 bp, synthetic construct
  misc_feature 1..19: note = Single strand DNA oligonucleotide
  source 1..19: mol_type = other DNA
cttccctaccttgggcacc

SEQ ID NO: 76 - DNA, 20 bp, synthetic construct
  misc_feature 1..20: note = Single strand DNA oligonucleotide
  source 1..20: mol_type = other DNA
atctgaactctgctgcactt

SEQ ID NO: 77 - DNA, 20 bp, synthetic construct
  misc_feature 1..20: note = Single strand DNA oligonucleotide
  source 1..20: mol_type = other DNA
tgtactgggcaaattcggtc

SEQ ID NO: 78 - DNA, 20 bp, synthetic construct
  misc_feature 1..20: note = Single strand DNA oligonucleotide
  source 1..20: mol_type = other DNA
gcaccaccgtctcccagccc

SEQ ID NO: 79 - DNA, 22 bp, synthetic construct
  misc_feature 1..22: note = Single strand DNA oligonucleotide
  source 1..22: mol_type = other DNA
gacgtttactccagcaatctgg

SEQ ID NO: 80 - DNA, 23 bp, synthetic construct
  misc_feature 1..23: note = Single strand DNA oligonucleotide
  source 1..23: mol_type = other DNA
gtaattggtaatctgataacaac

SEQ ID NO: 81 - DNA, 23 bp, synthetic construct
  misc_feature 1..23: note = Single strand DNA oligonucleotide
  source 1..23: mol_type = other DNA
atcctattgtgttatataaccac

SEQ ID NO: 82 - DNA, 21 bp, synthetic construct
  misc_feature 1..21: note = Single strand DNA oligonucleotide
  source 1..21: mol_type = other DNA
ttcagcatggatagtctgatc

SEQ ID NO: 83 - DNA, 21 bp, synthetic construct
  misc_feature 1..21: note = Single strand DNA oligonucleotide
  source 1..21: mol_type = other DNA
acgacggacttatagctagta

SEQ ID NO: 84 - DNA, 21 bp, synthetic construct
  misc_feature 1..21: note = Single strand DNA oligonucleotide
  source 1..21: mol_type = other DNA
gtatctgtctagtcacaacag

SEQ ID NO: 85 - DNA, 20 bp, synthetic construct
  misc_feature 1..20: note = Single strand DNA oligonucleotide
  source 1..20: mol_type = other DNA
gagtgctaacagaacatgcc

SEQ ID NO: 86 - DNA, 20 bp, synthetic construct
  misc_feature 1..20: note = Single strand DNA oligonucleotide
  source 1..20: mol_type = other DNA
gtccttcctcacctacgata

SEQ ID NO: 87 - DNA, 21 bp, synthetic construct
  misc_feature 1..21: note = Single strand DNA oligonucleotide
  source 1..21: mol_type = other DNA
gatactctggacttaacgtga

SEQ ID NO: 88 - protein, 10 aa, synthetic construct
  REGION 1..10: note = GnRH amino acid sequence
QHWSYGLRPG

SEQ ID NO: 89 - protein, 45 aa, synthetic construct
  REGION 1..45: note = minimal delivery protein (MDP) - chimeric protein (protamine andNLS)
RSQSRSRYYRQRQRSRRRRRRSGSGSSDDEATADAQHAAPPKKKR

SEQ ID NO: 90 - protein, 79 aa, synthetic construct
  REGION 1..79: note = Nur77 DBD amino acid sequence
GRCAVCGDNASCQHYGVRTCEGCKGFFKRTVQKSAKYICLANKDCPVDKRRRNRCQFCRFQKCLAVGMVKEVVRTDSLK

SEQ ID NO: 91 - DNA, 237 bp, synthetic construct
  misc_feature 1..237: note = Nur77 DBD nucleic acid sequence
  source 1..237: mol_type = other DNA
ggccgctgtgcagtctgtggtgacaatgcttcgtgtcagcactatggggtccgcacctgtgagggctgcaagggcttcttcaagcgcacagtacagaaaagcgccaagtacatctgcctggcaaacaaggattgccctgtggacaagaggcggcggaaccgctgccagttctgccgcttccagaagtgcctggctgtgggcatggtgaaggaagttgtacggacagacagcctaaaa

SEQ ID NO: 92 - protein, 191 aa, synthetic construct
  REGION 1..191: note = 92 HP1 alpha amino acid sequence
MGKKTKRTADSSSSEDEEEYVVEKVLDRRMVKGQVEYLLKWKGFSEEHNTWEPEKNLDCPELISEFMKKYKKMKEGENNKPREKSEGNKRKSSFSNSADDIKSKKKREQSNDIARGFERGLEPEKIIGATDSCGDLMFLMKWKDTDEADLVLAKEANVKCPQIVIAFYEERLTWHAYPEDAENKEKESAKS

SEQ ID NO: 93 - DNA, 573 bp, synthetic construct
  misc_feature 1..573: note = HP1 alpha nucleic acid sequence
  source 1..573: mol_type = other DNA
atgggaaagaagaccaagaggacagccgacagctcttcttcagaggatgaggaggaatatgtggtggaaaaggtgttggacaggcgcatggttaaggggcaagtggaatatctgttgaagtggaaaggcttttctgaggagcacaatacttgggaacctgagaagaacttggattgtcctgaactaatttctgagtttatgaaaaagtataagaagatgaaggagggtgaaaacaataagcccagggagaaatcagaaggaaacaagaggaaatccagtttctccaacagcgctgatgatattaaatctaaaaaaaagagagagcaaagcaatgatatcgctcggggctttgagagaggactggaaccagaaaagatcatcggagcaacagattcctgcggtgacttaatgttcttaatgaaatggaaagacacagatgaagctgacctggttcttgcaaaagaagctaacgtgaagtgtccacagattgtgatagcattttatgaagagagactgacgtggcacgcatatccagaggatgcggaaaacaaagaaaaagaaagcgcgaagagc

SEQ ID NO: 94 - protein, 908 aa, synthetic construct
  REGION 1..908: note = DNMT3a amino acid sequence
MPSSGPGDTSSSSLEREDDRKEGEEQEENRGKEERQEPSATARKVGRPGRKRKHPPVESSDTPKDPAVTTKSQPMAQDSGPSDLLPNGDLEKRSEPQPEEGSPAAGQKGGAPAEGEGTETPPEASRAVENGCCVTKEGRGASAGEGKEQKQTNIESMKMEGSRGRLRGGLGWESSLRQRPMPRLTFQAGDPYYISKRKRDEWLARWKREAEKKAKVIAVMNAVEENQASGESQKVEEASPPAVQQPTDPASPTVATTPEPVGGDAGDKNATKAADDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPEEEKNPYKEVYTDMWVEPEAAAYAPPPPAKKPRKSTTEKPKVKEIIDERTRERLVYEVRQKCRNIEDICISCGSLNVTLEHPLFIGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 95 - DNA, 2724 bp, synthetic construct
  misc_feature 1..2724: note = DNMT3a nucleic acid sequence
  source 1..2724: mol_type = other DNA
atgccctccagcggccccggggacaccagcagctcctctctggagcgggaggatgatcgaaaggaaggagaggaacaggaggagaaccgtggcaaggaagagcgccaggagcccagcgccacggcccggaaggtggggaggcctggccggaagcgcaagcacccaccggtggaaagcagtgacacccccaaggacccagcagtgaccaccaagtctcagcccatggcccaggactctggcccctcagatctgctacccaatggagacttggagaagcggagtgaaccccaacctgaggaggggagcccagctgcagggcagaagggtggggccccagctgaaggagagggaactgagaccccaccagaagcctccagagctgtggagaatggctgctgtgtgaccaaggaaggccgtggagcctctgcaggagagggcaaagaacagaagcagaccaacatcgaatccatgaaaatggagggctcccggggccgactgcgaggtggcttgggctgggagtccagcctccgtcagcgacccatgccaagactcaccttccaggcaggggacccctactacatcagcaaacggaaacgggatgagtggctggcacgttggaaaagggaggctgagaagaaagccaaggtaattgcagtaatgaatgctgtggaagagaaccaggcctctggagagtctcagaaggtggaggaggccagccctcctgctgtgcagcagcccacggaccctgcttctccgactgtggccaccacccctgagccagtaggaggggatgctggggacaagaatgctaccaaagcagccgacgatgagcctgagtatgaggatggccggggctttggcattggagagctggtgtgggggaaacttcggggcttctcctggtggccaggccgaattgtgtcttggtggatgacaggccggagccgagcagctgaaggcactcgctgggtcatgtggttcggagatggcaagttctcagtggtgtgtgtggagaagctcatgccgctgagctccttctgcagtgcattccaccaggccacctacaacaagcagcccatgtaccgcaaagccatctacgaagtcctccaggtggccagcagccgtgccgggaagctgtttccagcttgccatgacagtgatgaaagtgacagtggcaaggctgtggaagtgcagaacaagcagatgattgaatgggccctcggtggcttccagccctcgggtcctaagggcctggagccaccagaagaagagaagaatccttacaaggaagtttacaccgacatgtgggtggagcctgaagcagctgcttacgccccacccccaccagccaagaaacccagaaagagcacaacagagaaacctaaggtcaaggagatcattgatgagcgcacaagggagcggctggtgtatgaggtgcgccagaagtgcagaaacatcgaggacatttgtatctcatgtgggagcctcaatgtcaccctggagcacccactcttcattggaggcatgtgccagaactgtaagaactgcttcttggagtgtgcttaccagtatgacgacgatgggtaccagtcctattgcaccatctgctgtggggggcgtgaagtgctcatgtgtgggaacaacaactgctgcaggtgcttttgtgtcgagtgtgtggatctcttggtggggccaggagctgctcaggcagccattaaggaagacccctggaactgctacatgtgcgggcataagggcacctatgggctgctgcgaagacgggaagactggccttctcgactccagatgttctttgccaataaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtg

SEQ ID NO: 96 - protein, 4 aa, synthetic construct
  REGION 1..4: note = substrate for cleavage
  SITE 1: note = MISC_FEATURE - t-Butyloxycarbonyl (Boc) conjugated
  SITE 4: note = MISC_FEATURE - 4-methylcoumaryl-7-amide (MCA) conjugated
RVRR

SEQ ID NO: 97 - protein, 4 aa, synthetic construct
  REGION 1..4: note = cleavage motif
RVRR

SEQ ID NO: 98 - protein, 4 aa, synthetic construct
  REGION 1..4: note = cleavage motif
PRGW

SEQ ID NO: 99 - protein, 11 aa, synthetic construct
  REGION 1..11: note = cleavage motif
GGSGRSANAKC

SEQ ID NO: 100 - protein, 15 aa, synthetic construct
  REGION 1..15: note = part of the cell penetrating peptide NF51 SEQUENCE
  MOD_RES 1: note = Orn
  REGION 1..15: note = MISC_FEATURE - the cell-penetrating peptides was made by coupling Gly6 of Seq ID103to the Delta-NH2 group of ornithine 1
XINLKALAALAKKIL

SEQ ID NO: 101 - protein, 16 aa, synthetic construct
  REGION 1..16: note = part of the cell penetrating peptide NF51+CYSTEINE (PEPTIDE 1)
  REGION 1..16: note = MISC_FEATURE - the cell-penetrating peptides was made by coupling Gly6 of Seq ID103to the Delta-NH2 group of ornithine 1
  MOD_RES 1: note = Orn
  MOD_RES 16: note = C-terminal amide
XINLKALAALAKKILC

SEQ ID NO: 102 - protein, 20 aa, synthetic construct
  REGION 1..20: note = part of the cell penetrating peptide NF51+PACE CLEAVAGEMOTIF+CYSTEINE (PEPTIDE 2)
  REGION 1..20: note = MISC_FEATURE - the cell-penetrating peptides was made by coupling Gly6 of Seq ID103to the Delta-NH2 group of ornithine 1
  MOD_RES 1: note = Orn
  MOD_RES 20: note = C-terminal amide
XINLKALAALAKKILRVRRC

SEQ ID NO: 103 - protein, 6 aa, synthetic construct
  REGION 1..6: note = PART OF NF51 (cell penetrating peptide)
  MOD_RES 1: note = N-terminal stearic acid moiety
AGYLLG